SEQUENCE LISTING (5 sequences)

SEQ ID NO: 1 - DNA, 1320 bp, Homo sapiens
atggcagaaaataatgaaaatattagtaaaaatgtagatgtaaggcccaaaactagtcggagcagaagtgccgacagaaaagacggttatgtgtggagtggaaagaagttatcttggtcaaaaaagagtgagagttattcagatgctgagacagtgaatggtatagagaaaaccgaagtgtctttaaggaaccaagaaaggaagcacagctgttcatccattgagttggacttagatcattcctgtgggcatcgatttttaggccgatctcttaaacagaaactgcaagatgccgtggggcagtgttttccaataaagaattgtagtagtcggcactcttcagggcttccgtctaaaaggaaaattcatatcagtgaactcatgttagataagtgtcctttcccacctcgatcagatttagcctttaggtggcattttattaaacgacacactgctcctataaattccaaatcagatgaatgggtaagcacagacttgtctcagactgaattgagggatggtcagctaaaacgaagaaatatggaagaaaatataaactgtttctcacataccaatgttcagccctgtgtcataaccaccgacaatgctttgtgtagagaaggtcctatgactggctctgtgatgaacctggtttcaaataacagtatagaagatagtgatatggattccgatgatgaaattctaacactttgcacaagttccagaaaaagaaacaaacccaaatgggatttggatgatgaaatcctgcagttggaaacacctcctaaataccacacgcagattgattatgtccactgtcttgtaccagacctccttcagatcaataacaacccatgttactggggagtgatggataaatacgcagccgaagcactactggaaggaaaaccagagggtacctttttacttcgagactcagcacaggaagactatttattctctgttagttttagacgctatagtcgttctcttcatgctagaattgaacagtggaatcacaactttagctttgatgcacatgacccctgtgtcttccattctcctgacattactgggctcctagaacattataaggacccaagcgcctgtatgttctttgaaccacttctatccactcccttaattcggactttccctttttccctgcagcatatatgcagaacagttatttgtaactgtacaacttatgatggcatcgatgcccttccaattccttcttctatgaaattatatctgaaggaatatcattataaatcaaaagttagagtactcaggattgatgcaccagaacagcaatgc

SEQ ID NO: 2 - protein, 440 aa, Homo sapiens
MAENNENISKNVDVRPKTSRSRSADRKDGYVWSGKKLSWSKKSESYSDAETVNGIEKTEVSLRNQERKHSCSSIELDLDHSCGHRFLGRSLKQKLQDAVGQCFPIKNCSSRHSSGLPSKRKIHISELMLDKCPFPPRSDLAFRWHFIKRHTAPINSKSDEWVSTDLSQTELRDGQLKRRNMEENINCFSHTNVQPCVITTDNALCREGPMTGSVMNLVSNNSIEDSDMDSDDEILTLCTSSRKRNKPKWDLDDEILQLETPPKYHTQIDYVHCLVPDLLQINNNPCYWGVMDKYAAEALLEGKPEGTFLLRDSAQEDYLFSVSFRRYSRSLHARIEQWNHNFSFDAHDPCVFHSPDITGLLEHYKDPSACMFFEPLLSTPLIRTFPFSLQHICRTVICNCTTYDGIDALPIPSSMKLYLKEYHYKSKVRVLRIDAPEQQC

SEQ ID NO: 3 - DNA, 3001 bp, Homo sapiens
ttttgtatttttagtagagatggggtttcactgtgttggccaggctgggctggtcatgaactcctgacctcaggtaatccgcctgcctcagcctctcaaagtgctagtattaccacctcccctggcaaccctaaatcttattctgcataagaatacttaagtaaattccatttacaaacaatcattatcattagtcttcatgaaactcggtattttacattgcaaattctagtatgttttaccaacttcagaggaaaggcagtgccttatagaaatttatctgcttgttttcaaatgatcaagaatacaaatgagtatgtgtttaaaatggctaatttttatcaattttgtattttctaaaaattgcctagactaatggtaaagttgactaattattgaagtgagtttaaaacactgtattgggaatgcttactgagcatttttgctgttcaatggtgattgtttatttaataaattcttacagctttacaacttaaaattgttcatattttaatcagtagggaaggaaatagggagaagaaggtgaaggaactatgtagggtagtagttctcaaataccagctcatagatttgtgtctgtcttctacaaataattcactggtctgctgtgaaataagaaaactatggtaaggagagatgagcataagatatacttcaagactagaaactattcacatttgagtatttattatggttgatagaggaggcttggctttttcttatccacactgaaaattagctatgagaaaggaactcactcttcactctatggcactattaagttatcatgtatattatataatatatagcatgttatttaagtgggaacaaagtgtcttgggactcccttccaacatctctttttcatgtactaactgtgcttttccctcttgtgtctaatagaagcttaatatgtaaactagacaaggtagatgacaaatggttaatgacttttttttgttttctttagatacatccagaaagtgcccagaagaaacttcctgctggaaaaaatgaaaaagcagtatttataacattagaatctggataatttgttaacatggcagaaaataatgaaaatattagtaaaaatgtagatgtaaggcccaaaactagtcggagcagaagtgccgacagaaaagacggttatgtgtggagtggaaagaagttatcttggtcaaaaaagagtgagagttattcagatgctgagacagtgaatggtatagagaaaaccgaagtgtctttaaggaaccaagaaaggaagcacagctgttcatccattgagttggacttagatcattcctgtgggcatcgatttttaggccgatctcttaaacagaaactgcaagatgccgtggggcagtgttttccaataaagaattgtagtagtcggcactcttcagggcttccgtctaaaaggaaaattcatatcagtgaactcatgttagataagtgtcctttcccacctcgatcagatttagcctttaggtggcattttattaaacgacacactgctcctataaattccaaatcagatgaatgggtaagcacagacttgtctcagactgaattgagggatggtcagctaaaacgaagaaatatggaagaaaatataaactgtttctcacataccaatgttcagccctgtgtcataaccaccgacaatgctttgtgtagagaaggtcctatgactggctctgtgatgaacctggtttcaaataacagtatagaagatagtgatatggattccgatgatgaaattctaacactttgcacaagttccagaaaaagaaacaaacccaaatgggatttggatgatgaaatcctgcagttggaaacacctcctaaataccacacgcagattgattatgtccactgtcttgtaccagacctccttcagatcaataacaacccatgttactggggagtgatggataaatacgcagccgaagcactactggaaggaaaaccagagggtacctttttacttcgagactcagcacaggaagactatttattctctgttagttttagacgctatagtcgttctcttcatgctagaattgaacagtggaatcacaactttagctttgatgcacatgacccctgtgtcttccattctcctgacattactgggctcctagaacattataaggacccaagcgcctgtatgttctttgaaccacttctatccactcccttaattcggactttccctttttccctgcagcatatatgcagaacagttatttgtaactgtacaacttatgatggcatcgatgcccttccaattccttcttctatgaaattatatctgaaggaatatcattataaatcaaaagttagagtactcaggattgatgcaccagaacagcaatgctagtaacaggatgggaacatgggaatgataatatatattttttcttttaatattttatttttctttttatgccactttggatttttctacaaaggcagtggtgtccaaaataaaatctctgccctaaattttactaataaatccatttttctagtgatacacaaattgtttaaggttatacactcgagcttaaatagatatttttaaccaggtgtttggtttttgtttttaccgtgtaggttgtatacttacattttttctttccttaatttatacatgatcctttttccttaatttatacatgatccaggcacatttgaaatttggtaggcattgcaaacacatttgaatattgtgtatttcatactattctttaaaagtaatcttatgtcctttcctacatgtaaaatattttgttaatctatgccattagtagtattatatataaaatatagttccccgtcccccttttcattgagtttgatattcttcagtaaagctgaatgttgtaattcaccattcatactaatgttattgacttttgtaacttactaatagggatgttaaaagtaacaaaaccaagtcaaacttggtgtattt

SEQ ID NO: 4 - DNA, 18 bp, synthetic construct
atggcagaaaataatgaa

SEQ ID NO: 5 - DNA, 18 bp, synthetic construct
ctagcattgctgttctgg

(19) United States
(12) Patent Application Publication
(10) Pub. No.: US 2004/0106784 A1
(43) Pub. Date: Jun. 3, 2004

(54) Socs (suppressor of cytokine signaling)

(76) Inventor: Luis A. Parodi, London (GB)

Correspondence Address: PHARMACIA & UPJOHN, 301 HENRIETTA ST, 0228-32-LAW, KALAMAZOO, MI, 49007, US

(21) Appl. No.: 10/451,410

(22) PCT Filed: Dec. 7, 2001
(86) PCT No.: PCT/US01/44697

(51) Int. Cl. C07H 21/04; C07K 14/47

(52) U.S. Cl. 536/23.5; 530/350; 435/69.1; 435/320.1; 435/325

(57) ABSTRACT

The present invention provides a cDNA encoding a heretofore unknown polypeptide termed SOCS-8; constructs and recombinant host cells incorporating the cDNA; the SOCS-8 polypeptide encoded by the gene; antibodies to the polypeptide; and methods of making and using all of the foregoing.

### CROSS REFERENCE TO RELATED APPLICATIONS

The present application claims priority of U.S. Application Serial No. 60/255,576 filed 14 Dec. 2000 which is hereby incorporated by reference.

### BACKGROUND OF THE INVENTION

1. Field of the Invention

The present invention provides an isolated SOCS (Suppressor of Cytokine Signaling) polypeptide, and the isolated polynucleotide molecules that encode them, as well as vectors and host cells comprising such polynucleotide molecules. The invention also provides methods for the identification of an agent that alters SOCS activity as well as antibodies specifically reactive with the SOCS polypeptide.

2. Description of Related Art

Regulation of many aspects of cell behavior occurs through the interaction of cytokines with specific cell surface receptors, resulting in the activation of cytoplasmic signal transduction pathways. Cellular responses to cytokines are tightly controlled, however only a few molecules have been identified which are able to switch these signals off. The suppressors of cytokine signaling (SOCS) proteins are a family of negative regulators of cytokine signal transduction. SOCS proteins contain a variable amino terminal region, a central Src-homology 2 (SH2) domain and a conserved carboxy terminal motif called the SOCS box. The expression of SOCS proteins are induced by cytokines. Once expressed, SOCS down regulate JAK/STAT pathways and concomitantly the biological response to the cytokine. Recent studies primarily reliant on over expression of proteins, indicate that SOCS may be involved in modulating additional pathways, suggesting that SOCS may play a more general role in the cytokine signalling process. Mutations leading to loss of SOCS activity may give rise to cytokine hyperesponsiveness and may contribute to the development of diseases such as diabetes and cancer. Small molecule effectors that modify SOCS function are useful therapeutics for the treatment of certain diseases including inflammatory disorders, metabolic disorders, growth disorders and cancers.

### REFERENCES CITED

Patent Documents

1. EP-A-0367566; and WO 91/18982, Type II Interleukin-1 Receptors

2. WO 94/12650, Activating Expression of an Amplifying Endogenous Gene by Homologous Recombination

3. WO 92/20808, Genomic Modifications with Homologous DNA Targeting

4. WO 91/09955, Endogenous Gene Expression Modification with Regulatory Element

5. WO 97/09433, Cell-Cycle Checkpoint Genes

6. WO93/11236, Production of Anti-Self Antibodies From Antibody Segment Repertoires and Displayed on Phage

Non-Patent Documents

1. Anderson, W. F., Human gene therapy. Nature, 1998.392(6679 Suppl): p. 25-30.

2. Aujame, L., F. Geoffroy, and R. Sodoyer, High affinity human antibodies by phage display. Hum Antibodies, 1997.8(4): p. 155-68.

3. Bruggemann, M. and M. S. Neuberger, Strategies for expressing human antibody repertoires in transgenic mice. Immunol Today, 1996.17(8): p. 391-7.

4. Bruggemann, M. and M. J. Taussig, Production of human antibody repertoires in transgenic mice. Curr Opin Biotechnol, 1997. 8(4): p. 455-8.

5. Capecchi, M. R., Altering the genome by homologous recombination. Science, 1989.244(4910): p. 1288-92.

6. Choo, Y., et al., Promoter-specific activation of gene expression directed by bacteriophage-selected zinc fingers. J Mol Biol, 1997.273(3): p. 525-32.

7. Cosman, D., et al., Cloning, sequence and expression of human interleukin-2 receptor. Nature, 1984.312(5996): p. 768-71.

8. Cosman, D., et al., High level stable expression of human interleukin-2 receptors in mouse cells generates only low affinity interleukin-2 binding sites. Mol Immunol, 1986.23(9): p. 935-41.

9. Dayhoff, in Atlas of Protein Sequence and Structure, Vol. 5, p. 124, National Biochemical Research Foundation, Washington, D.C. (1972)

10. Erlich H. A., ed., PCR Technology, Stockton Press, New York, 1989;

11. Foote, J. and G. Winter, Antibody framework residues affecting the conformation of the hypervariable loops. J Mol Biol, 1992.224(2): p. 487-99.

12. Gluzman, Y., SV40-transformed simian cells support the replication of early SV40 mutants. Cell, 1981.23(1): p. 175-82.

13. Greisman, H. A. and C. O. Pabo, A general strategy for selecting high-affinity zinc finger proteins for diverse DNA target sites. Science, 1997. 275(5300): p. 657-61.

14. Harlow et al. (Eds), Antibodies A Laboratory Manual; Cold Spring Harbor Laboratory; Cold Spring Harbor, N.Y. (1988), Chapter 6

15. Hoogenboom, H. R., Designing and optimizing library selection strategies for generating high-affinity antibodies. Trends Biotechnol, 1997.15(2): p. 62-70.

16. Innis M. A., et al. eds PCR Protocols: A Guide to Methods and Applications 1990, Academic Press, Inc., New York,

17. Jones, P. T., et al., Replacing the complementarity-determining regions in a human antibody with those from a mouse. Nature, 1986.321(6069): p. 522-5.

18. Kettleborough, C. A., et al., Humanization of a mouse monoclonal antibody by CDR-grafting: the importance of framework residues on loop conformation. Protein Eng, 1991. 4(7): p. 773-83.

19. Kim, J. S., et al., Design of TATA box-binding protein/zinc finger fusions for targeted regulation of gene expression. Proc Natl Acad Sci USA, 1997. 94(8): p. 3616-20.

20. Lehninger, Biochemistry, Second Edition; Worth Publishers, Inc. NY:N.Y. (1975), pp. 71-77

21. Liu, Q., et al., Design of polydactyl zinc-finger proteins for unique addressing within complex genomes. Proc Natl Acad Sci USA, 1997. 94(11): p. 5525-30.

22. Margalit, H., et al., Prediction of immunodominant helper T cell antigenic sites from the primary sequence. J Immunol, 1987. 138(7): p. 2213-29

23. McColl, D. J., C. D. Honchell, and A. D. Frankel, Structure-based design of an RNA-binding zinc finger. Proc Natl Acad Sci USA, 1999. 96(17): p. 9521-6.

24. Miller, A. D., Human gene therapy comes of age. Nature, 1992. 357(6378): p. 455-60.

25. Okayama, H. and P. Berg, A cDNA cloning vector that permits expression of cDNA inserts in mammalian cells. Mol Cell Biol, 1983. 3(2): p. 280-9.

26. 42. Rader, C. and C. F. Barbas, 3rd, Phage display of combinatorial antibody libraries. Curr Opin Biotechnol, 1997. 8(4): p. 503-8.

27. Riechmann, L., et al., Reshaping human antibodies for therapy. Nature, 1988. 332(6162): p. 323-7.

28. Sambrook, et al., (Eds.), Molecular Cloning: A Laboratory Manual, Cold Spring Harbor Laboratory Press: Cold Spring Harbor, N.Y. (1989).

29. Segal, D. J., et al., Toward controlling gene expression at will: selection and design of zinc finger domains recognizing each of the 5′-GNN-3′ DNA target sequences. Proc Natl Acad Sci USA, 1999. 96(6): p. 2758-63.

30. Tempest, P. R., et al., Reshaping a human monoclonal antibody to inhibit human respiratory syncytial virus infection in vivo. Biotechnology (N Y), 1991. 9(3): p. 266-71.

31. Verhoeyen, M., C. Milstein, and G. Winter, Reshaping human antibodies: grafting an antilysozyme activity. Science, 1988. 239(4847): p. 1534-6

32. Verma, I. M., Gene therapy. Sci Am, 1990. 263(5): p. 68-72, 81-4.

33. Vettel, U., et al., Coordinate secretion and functional synergism of T cell-associated serine proteinase-1 (MTSP-1) and endoglycosidase(s) of activated T cells. Eur J Immunol, 1991. 21(9): p. 2247-51.

34. Van Regenmortel, M. H. V., 1986. Trends in Biological Sciences 11: 36-39.

35. Wu, H., W. P. Yang, and C. F. Barbas, 3rd, Building zinc fingers by selection: toward a therapeutic application. Proc Natl Acad Sci USA, 1995. 92(2): p. 344-8.

### SUMMARY OF THE INVENTION

The present invention addresses the need identified above in that it provides isolated nucleic acid molecules encoding a heretofore unknown suppressor of cytokine signalling termed SOCS-8; constructs and recombinant host cells incorporating the isolated nucleic acid molecules; the SOCS-8 polypeptides encoded by the isolated nucleic acid molecules; antibodies to the SOCS-8 polypeptide; and methods of making and using all of the foregoing.

In one embodiment, the invention provides an isolated SOCS-8 polypeptide comprising the amino acid sequence set forth in SEQ ID NO: 2. It is understood that the polypeptide of SEQ ID NO:2 may be subject to specific proteolytic processing events resulting in a number of polypeptide species

In addition the invention provides a fragment comprising an epitope of the SOCS-8 polypeptide. By “epitope specific to” is meant a portion of the SOCS-8 enzyme that is recognizable by an antibody that is specific for SOCS-8 polypeptide, as defined in detail below. Another embodiment comprises an isolated polypeptide comprising the complete amino acid sequence set forth in SEQ ID NO: 2. The cDNA sequence and predicted amino acid sequence of human SOCS-8 is reproduced below.

ATGGCAGAAAATAATGAAAATATTAGTAAAAATGTAGATGTAAGGCCCAAAACTAGTCGG⁶⁰
M A E N N E N I S K N V D V R P K T S R
AGCAGAAGTGCCGACAGAAAAGACGGTTATGTGTGGAGTGGAAAGAAGTTATCTTGGTCA¹²⁰
S R S A D R K D G Y V W S G K K L S W S
AAAAAGAGTGAGAGTTATTCAGATGCTGAGACAGTGAATGGTATAGAGAAAACCGAAGTG¹⁸⁰
K K S E S Y S D A E T V N G I E K T E V
TCTTTAAGGAACCAAGAAAGGAAGCACAGCTGTTCATCCATTGAGTTGGACTTAGATCAT²⁴⁰
S L R N Q E R K H S C S S I E L D L D H
TCCTGTGGGCATCGATTTTTAGGCCGATCTCTTAAACAGAAACTGCAAGATGCCGTGGGG³⁰⁰
S C G H R F L G R S L K Q K L Q D A V G
CAGTGTTTTCCAATAAAGAATTGTAGTAGTCGGCACTCTTCAGGGCTTCCGTCTAAAAGG³⁶⁰
Q C F P I K N C S S R H S S G L P S K R
AAAATTCATATCAGTGAACTCATGTTAGATAAGTGTCCTTTCCCACCTCGATCAGATTTA⁴²⁰
K I H I S E L M L D K C P F P P R S D L
GCCTTTAGGTGGCATTTTATTAAACGACACACTGCTCCTATAAATTCCAAATCAGATGAA⁴⁸⁰
A F R W H F I K R H T A P I N S K S D E
TGGGTAAGCACAGACTTGTCTCAGACTGAATTGAGdGATGGTCAGCTAAAACGAAGAAAT⁵⁴⁰
W V S T D L S Q T E L R D G Q L K R R N
ATGGAAGAAAATATAAACTGTTTCTCACATACCAATGTTCAGCCCTGTGTCATAACCACC⁶⁰⁰
M E E N I N C F S H T N V Q P C V I T T
GACAATGCTTTGTGTAGAGAAGGTCCTATGACTGGCTCTGTGATGAACCTGGTTTCAAAT⁶⁶⁰
D N A L C R E G P H T G S V H N L V S N
AACAGTATAGAAGATAGTGATATGGATTCCGATGATGAAATTCTAACACTTTGCACAAGT⁷²⁰
N S I E D S D M D S D D E I L T L C T S
TCCAGAAAAAGAAACAAACCCAAATGGGATTTGGATGATGAAATCCTGCAGTTGGAAACA⁷⁸⁰
S R K R N K P K W D L D D E I L Q L E T
CCTCCTAAATACCACACGCAGATTGATTATGTCCACTGTCTTGTACCAGACCTCCTTCAG⁸⁴⁰
P P K Y H T Q I D Y V H C L V P D L L Q
ATCAATAACAACCCATGTTACTGGGGAGTGATGGATAAATACGCAGCCGAAGCACTACTG⁹⁰⁰
I N N N P C Y W G V H D K Y A A E A L L
GAAGGAAAACCAGAGGGTACCTTTTTACTTCGAGACTCAGCACAGGAAGACTATTTATTC⁹⁶⁰
E G K P E G T E L L R D S A Q E D Y L F
TCTGTTAGTTTTAGACGCTATAGTCGTTCTCTTCATGCTAGAATTGAACAGTGGAATCAC¹⁰²⁰
S V S F R R Y S R S L H A R I E Q W N H
AACTTTAGCTTTGATGCACATGACCCCTGTGTCTTCCATTCTCCTGACATTACTGGGCTC¹⁰⁸⁰
N F S F D A N D P C V F H S P D I T G L
CTAGAACATTATAAGGACCCAAGCGCCTGTATGTTCTTTGAACCACTTCTATCCACTCCC¹¹⁴⁰
L E H Y K D P S A C M F F E P L L S T P
TTAATTCGGACTTTCCCTTTTTCCCTGCAGCATATATGCAGAACAGTTATTTGTAACTGT¹²⁰⁰
L I R T F P F S L Q H I C R T V I C N C
ACAACTTATGATGGCATCGATGCCCTTCCAATTCCTTCTTCTATGAAATTATATCTGAAG¹²⁶⁰
T T Y D G I D A L P I P S S M K L Y L K
GAATATCATTATAAATCAAAAGTTAGAGTACTCAGGATTGATGCACCAGAACAGCAATGC¹³²⁰
E Y H Y K S K V R V L R I D A P E Q Q C
TAG¹³²³
*

Although SEQ ID NOS: 1 and 2 provides particular human polynucleotide and polypeptide sequences, the invention is intended to include within its scope other human allelic variants; non-human mammalian forms of SOCS-8, and other vertebrate forms of SOCS-8.

In another embodiment, the invention provides isolated polynucleotides (e.g., cDNA, genomic DNA, synthetic DNA, RNA, or combinations thereof, single or double stranded) that comprise a nucleotide sequence encoding the amino acid sequence of the polypeptides of the invention. A genomic sequence encoding SOC-8 is included herein as SEQ ID NO:3. Such polynucleotides are useful for recombinantly expressing the enzyme and also for detecting expression of the enzyme in cells (e.g., using Northern hybridization and in situ hybridization assays). Such polynucleotides also are useful to design antisense and other molecules for the suppression of the expression of SOCS-8 in a cultured cell or tissue or in an animal, for therapeutic purposes or to provide a model for diseases characterized by aberrant SOCS-8 expression. Specifically excluded from the definition of polynucleotides of the invention are entire isolated chromosomes from native host cells from which the polynucleotide was originally derived. The polynucleotide set forth in SEQ ID NO: 1 corresponds to naturally occurring a SOCS-8 sequence. It will be appreciated that numerous other sequences exist that also encode SOCS-8 of SEQ ID NO: 2 due to the well-known degeneracy of the universal genetic code. In another embodiment, the invention is directed to all of the degenerate SOCS-8-encoding sequences other than the sequence set forth in SEQ ID NO: 1.

The invention also provides an isolated polynucleotide comprising a nucleotide sequence that encodes a mammalian SOCS-8, wherein the polynucleotide hybridizes to the nucleotide sequence set forth in SEQ ID NO: 1 or 3 or the non-coding strand complementary thereto, under the following hybridization conditions:

(a) hybridization for 16 hours at 42° C. in a hybridization solution comprising 50% formamide, 1% SDS, 1M NaCl, 10% Dextran sulfate; and

(b) washing 2 times for 30 minutes at 60° C. in a wash solution comprising 0.1% SSC, 1% SDS.

One polynucleotide of the invention comprises the sequence set forth in SEQ ID NO: 1, which comprises a human SOCS-8 encoding DNA sequence: Another sequence of the invention includes the genomic sequence set forth in SEQ ID NO:3.

In a related embodiment, the invention provides vectors comprising a polynucleotide of the invention. Such vectors are useful, e.g., for amplifying the polynucleotides in host cells to create useful quantities thereof. In other embodiments, the vector is an expression vector wherein the polynucleotide of the invention is operatively linked to a polynucleotide comprising an expression control sequence. Such vectors are useful for recombinant production of polypeptides of the invention.

In another related embodiment, the invention provides host cells that are transformed or transfected (stably or transiently) with polynucleotides of the invention or vectors of the invention. As stated above, such host cells are useful for amplifying the polynucleotides and also for expressing the SOCS-8 polypeptide or fragment thereof encoded by the polynucleotide.

In still another related embodiment, the invention provides a method for producing a SOCS-8 polypeptide (or fragment thereof) comprising the steps of growing a host cell of the invention in a nutrient medium and isolating the polypeptide or variant thereof from the cell or the medium.

In still another embodiment, the invention provides an antibody that is specific for the SOCS-8 of the invention. Antibody specificity is described in greater detail below. However, it should be emphasized that antibodies that can be generated from polypeptides that have previously been described in the literature and that are capable of fortuitously cross-reacting with SOCS-8 (e.g., due to the fortuitous existence of a similar epitope in both polypeptides) are considered “cross-reactive” antibodies. Such cross-reactive antibodies are not antibodies that are “specific” for SOCS-8. The determination of whether an antibody is specific for SOCS-8 or is cross-reactive with another known enzyme is made using Western blotting assays or several other assays well known in the literature. For identifying cells that express SOCS-8 and also for modulating SOCS-8 activity, antibodies that specifically bind to the active site of SOCS-8 are particularly useful but of course, antibodies binding other epitopes are contemplated as part of the invention as well.

In one variation, the invention provides monoclonal antibodies. Hybridomas that produce such antibodies also are intended as aspects of the invention. In yet another variation, the invention provides a humanized antibody. Humanized antibodies are useful for in vivo therapeutic indications.

In another variation, the invention provides a cell-free composition comprising polyclonal antibodies, wherein at least one of the antibodies is an antibody of the invention specific for SOCS-8. Antisera isolated from an animal is an exemplary composition, as is a composition comprising an antibody fraction of an antisera that has been resuspended in water or in another diluent, excipient, or carrier.

The invention also provides methods of using antibodies of the invention. For example, the invention provides a method for determining the amount of SOC-8 present within a cellular extract comprising the step of contacting SOCS-8 polypeptide with an antibody specific for the SOCS-8 polypeptide, under conditions wherein the antibody binds the SOCS-8 polypeptide

The invention also provides assays to identify compounds that modulate SOCS-8 biologic activity. One such assay comprises the steps of contacting a SOCS-8 polypeptide and a binding partner polypeptide the presence and absence of a test agent; determining the propensity of the SOCS-8 polypeptide to associate with the binding partner polypeptide in the presence and absence of the test agent; and comparing the propensity of the SOCS-8 polypeptide to associate with the binding partner polypeptide in the presence and the absence of the test agent. Such an assay can be performed with isolated SOCS-8 polypeptide or may be performed with SOCS-8 within a cell. Compounds identified by such an assay have use in treating a disease state including those caused by cytokine hyperesponsiveness including diabetes, as well as inflammatory disorders, metabolic disorders, growth disorders and cancers.

The invention also provides a method for treating such a disease state comprising the step of administering to a mammal in need of such treatment an amount of an agent sufficient to alter SOCS-8 activity in the tissues of said mammal

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE FIGURES

FIG. 1 Amino acid sequence of SOCS-8 with structural motifs delineated. The SH2 domain is underlined and the SOCS box is italicized and bolded.

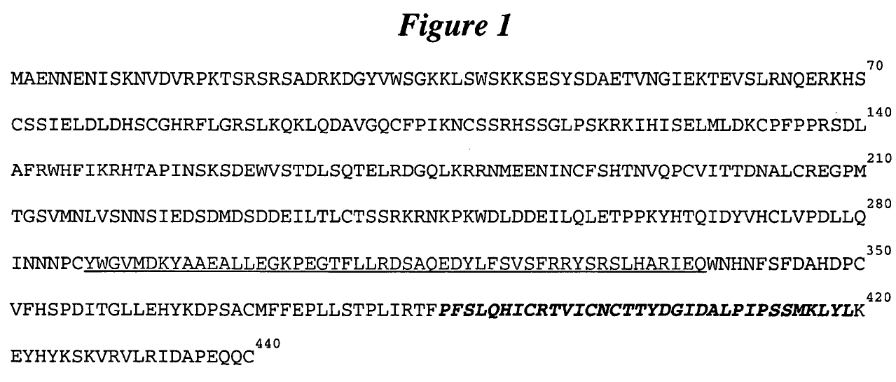

### BRIEF DESCRIPTION OF THE SEQUENCE LISTINGS

SEQ ID NO: 1-cDNA sequence encoding human SOCS-8

SEQ ID NO: 2-predicted amino acid sequence of SOCS-8

SEQ ID NO:3—genomic sequence containing the SOCS-8 sequence

SEQ ID NO:4——PCR primer

SEQ ID NO:5——PCR primer

### DETAILED DESCRIPTION OF THE INVENTION

The present invention provides isolated polynucleotides (e.g., DNA sequences and RNA transcripts, both sense and complementary antisense strands, both single and double-stranded, including splice variants thereof) encoding an SOCS-8 polypeptide referred to herein as SOCS-8. DNA polynucleotides of the invention include genomic DNA, cDNA, and DNA that has been chemically synthesized in whole or in part.

General Definitions

As used hereinafter “isolated” means altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

As used hereinafter “polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

As used hereinafter “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation prenylation, racemization, selenoylation, sulfation, transfer-RNA mediated addition of amino acids to proteins such as arginylation, and ubiquitination (see, for instance, Proteins-Structure and Molecular Properties, 2nd Ed., T. E. Creighton, W. H. Freeman and Company, New York, 1993; Wold, F., Post-translational Protein Modifications: Perspectives and Prospects, pgs. 1-12 in Postranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Post-translational Modifications and Aging”, Ann NY Acad Sci (1992) 663:4842).

“Synthesized” as used herein and understood in the art, refers to polynucleotides or polypeptides produced by purely chemical, as opposed to enzymatic, methods. “Wholly” synthesized DNA or protein sequences are therefore produced entirely by chemical means, and “partially” synthesized DNAs or proteins embrace those wherein only portions of the resulting DNA were produced by chemical means.

“Isolated” as used herein and as understood in the art, whether referring to “isolated” polynucleotides or polypeptides, is taken to mean separated from the original cellular environment in which the polypeptide or nucleic acid is normally found. As used herein therefore, by way of example only, a transgenic animal or a recombinant cell line constructed with a polynucleotide of the invention, makes use of the “isolated” nucleic acid.

As used herein, the term “contacting” means bringing together, either directly or indirectly, a compound into physical proximity to a polypeptide or polynucleotide of the invention. Additionally “contacting” may mean bringing a polypeptide of the invention into physical proximity with another polypeptide or polynucleotide (either another polypeptide or polynucleotide of the invention or a polypeptide or polynucleotide not so claimed) or bringing a polynucleotide of the invention into physical proximity with a polypeptide or polynucleotide (either a polypeptide or polynucleotide of the invention or a polypeptide or polynucleotide not so claimed).

As used herein “binding partner polypeptide” refers to a polypeptide species capable of interacting with a SOCS-8 polypeptide where the interaction is sufficiently strong to permit measurement of their association. Methods of accessing the propensity of a polypeptide to associate with another polypeptide are well known in the art and illustrative examples are discussed.

### Polynucleotides of the Invention

The present invention provides isolated polynucleotides (e.g., DNA sequences and RNA transcripts, both sense and complementary antisense strands, both single and double-stranded, including splice variants thereof) encoding an SOCS-8 polypeptide referred to herein as SOCS-8. DNA polynucleotides of the invention include genomic DNA, cDNA, and DNA that has been chemically synthesized in whole or in part.

Genomic DNA of the invention comprises the protein coding region for a polypeptide of the invention and is also intended to include allelic variants thereof. It is widely understood that, for many genes, genomic DNA is transcribed into RNA transcripts that undergo one or more splicing events wherein intron (i.e., non-coding regions) of the transcripts are removed, or “spliced out.” RNA transcripts that can be spliced by alternative mechanisms, and therefore be subject to removal of different RNA sequences but still encode a SOCS-8 polypeptide, are referred to in the art as splice variants which are embraced by the invention. Splice variants comprehended by the invention therefore are encoded by the same original genomic DNA sequences but arise from distinct mRNA transcripts. Allelic variants are modified forms of a wild type gene sequence, the modification resulting from recombination during chromosomal segregation or exposure to conditions which give rise to genetic mutation. Allelic variants, like wild type genes, are naturally occurring sequences (as opposed to non-naturally occurring variants which arise from in vitro manipulation).

The invention also comprehends cDNA that is obtained through reverse transcription of an RNA polynucleotide (conventionally followed by second strand synthesis of a complementary strand to provide a double-stranded DNA) encoding a SOCS-8 polypeptide A DNA sequence encoding a human SOCS-8 polypeptide is set out in SEQ ID NO: 1 with a corresponding genomic sequence set forth in SEQ ID NO:3. The worker of skill in the art will readily appreciate that the DNA of the invention comprises a double stranded molecule, for example the molecule having the sequence set forth in SEQ ID NO: 1 or 3 along with the complementary molecule (the “non-coding strand” or “complement”) having a sequence deducible from the sequence of SEQ ID NO: 1 according to Watson-Crick base pairing rules for DNA. Also contemplated by the invention are other polynucleotides encoding the SOCS-8 polypeptide of SEQ ID NO: 2, which differ in sequence from the polynucleotide of SEQ ID NO: 1 or 3 by virtue of the well-known degeneracy of the universal genetic code.

As is well known in the art, due to the degeneracy of the genetic code, there are numerous other DNA and RNA molecules that can code for the same polypeptide as that encoded by the aforementioned SEQ ID NO: 1 or 3 polynucleotides. The present invention, therefore, contemplates those other DNA and RNA molecules which, on expression, encode the polypeptides of SEQ ID NO: 2 Having identified the amino acid residue sequence encoded the SOCS-8 polypeptide, and with the knowledge of all triplet codons for each particular amino acid residue, it is possible to describe all such encoding RNA and DNA sequences. DNA and RNA molecules other than those specifically disclosed herein characterized simply by a change in a codon for a particular amino acid, are, therefore, within the scope of this invention.

A table of amino acids and their representative abbreviations, symbols and codons is set forth below in the following Table 1.

TABLE 1

| | | | |
|---|---|---|---|
| Amino acid | Abbrev. | Symbol | Codon(s) |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparaginc | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

As is well known in the art, codons constitute triplet sequences of nucleotides in mRNA and their corresponding cDNA molecules Codons are characterized by the base uracil (U) when present in a mRNA molecule but are characterized by base thymidine (T) when present in DNA. A simple change in a codon for the same amino acid residue within a polynucleotide will not change the sequence or structure of the encoded polypeptide., It is apparent that when a phrase stating that a particular 3 nucleotide sequence “encode(s)” any particular amino acid, the ordinarily skilled artisan would recognize that the table above provides a means of identifying the particular nucleotides at issue. By way of example, if a particular three nucleotide sequence encodes theonine the table above discloses that the posible triplet sequences are ACA, ACG, ACC and ACU (ACT if in DNA)

The invention further embraces species, preferably mammalian, homologs of the human SOCS-8 polynucleotide. Species homologs, sometimes referred to as “orthologs,” in general, share at least 35%, at least 40%, at least 45%, at least 50%, at least 60%, at least 65%, at least 70%, at least 75%, at least 80%, at least 85%, at least 90%, at least 95%, at least 98%, or at least 99% homology with SEQ ID NO: 1 of the invention. Percent sequence “homology” with respect to polynucleotides of the invention is defined herein as the percentage of nucleotide bases in the candidate sequence that are identical to nucleotides in the SOCS-8 sequence set forth in SEQ ID NO: 1, after aligning the sequences and introducing gaps, if necessary, to achieve the maximum percent sequence identity. The percentage of sequence between a native and a variant human SOCS sequence may also be determined, for example, by comparing the two sequences using any of the computer programs commonly employed for this purpose, such as the Gap program (Wisconsin Sequence Analysis Package, Version 8 for Unix, Genetics Computer Group, University Research Park, Madison Wis.), which uses the algorithm of Smith and Waterman (Adv. Appl. Math. 2: 482-489 (1981)).

The polynucleotide sequence information provided by the invention makes possible large scale expression of the encoded polypeptide by techniques well known and routinely practiced in the art. Polynucleotides of the invention also permit identification and isolation of polynucleotides encoding related SOCS-8 polypeptides, such as human allelic variants and species homologs, by well known techniques including Southern and/or Northern hybridization, and polymerase chain reaction (PCR). Examples of related polynucleotides include human and non-human genomic sequences, including allelic variants, as well as polynucleotides encoding polypeptides homologous to SOCS-8 and structurally related polypeptides sharing one or more biological, immunological, and/or physical properties of SOCS-8. Non-human species genes encoding proteins homologous to SOCS-8 can also be identified by Southern and/or PCR analysis and are useful in animal models for SOCS-8 disorders. Knowledge of the sequence of a human SOCS-8 DNA also makes possible through use of Southern hybridization or polymerase chain reaction (PCR) the identification of genomic DNA sequences encoding SOCS-8 expression control regulatory sequences such as promoters, operators, enhancers, repressors, and the like. Polynucleotides of the invention are also useful in hybridization assays to detect the capacity of cells to express SOCS-8. Polynucleotides of the invention may also be the basis for diagnostic methods useful for identifying a genetic alteration(s) in a SOCS-8 locus that underlies a disease state or states, which information is useful both for diagnosis and for selection of therapeutic strategies.

The disclosure herein of a full length polynucleotides encoding a SOCS-8 polypeptide makes readily available to the worker of ordinary skill in the art every possible fragment of the full length polynucleotide. The invention therefore provides fragments of SOCS-8-encoding polynucleotides comprising at least 14-15, and preferably at least 18, 20, 25, 50, or 75 consecutive nucleotides of a polynucleotide encoding SOCS-8. Preferably, fragment polynucleotides of the invention comprise sequences unique to the SOCS-8-encoding polynucleotide sequence, and therefore hybridize under highly stringent or moderately stringent conditions only (i.e., “specifically”) to polynucleotides encoding SOCS-8 (or fragments thereof). Polynucleotide fragments of genomic sequences of the invention comprise not only sequences unique to the coding region, but also include fragments of the full length sequence derived from introns, regulatory regions, and/or other non-translated sequences. Sequences unique to polynucleotides of the invention are recognizable through sequence comparison to other known polynucleotides, and can be identified through use of alignment programs routinely utilized in the art, e.g., those made available in public sequence databases. Such sequences also are recognizable from Southern hybridization analyses to determine the number of fragments of genomic DNA to which a polynucleotide will hybridize. Polynucleotides of the invention can be labeled in a manner that permits their detection, including radioactive, fluorescent, and enzymatic labeling.

Fragment polynucleotides are particularly useful as probes for detection of full length or other fragment SOCS-8 polynucleotides. One or more fragment polynucleotides can be included in kits that are used to detect the presence of a polynucleotide encoding SOCS-8, or used to detect variations in a polynucleotide sequence encoding SOCS-8.

The invention also embraces polynucleotides encoding SOCS-8 polypeptides which DNAs hybridize under moderately stringent or high stringency conditions to the non-coding strand, or complement, of the polynucleotide in SEQ ID NO: 1 and 3

Exemplary highly stringent hybridization conditions are as follows: hybridization at 42° C. in a hybridization solution comprising 50% formamide, 1% SDS, 1M NaCl, 10% Dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

### EXAMPLE 1

### Cloning of SOCS-8

The human genomic sequence data from Celera release 1.20 and 1.21 was searched using an HMM derived from the alignment of several SOCS-box domains and also with a Pfam HMM for the SH2 domain. Sequences containing both domains (potential novel SOCS) were analyzed. Several pseudogenes were eliminated and a single sequence (SEQ ID NO:3) was identified as a novel human SOCS with the coding sequence presented in SEQ ID NO: 1. The sequence of the SOCS gene may not be limited to SEQ ID NO:3 with 3001 nucleotides presented here.

Given the coding and flanking sequences obtained, primers are easily designed which flank or include the coding sequence and are suitable for PCR amplification. Methods for preparing and using probes and primers are described, for example, in Sambrook et al., Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1987 (with periodic updates); and Innis et al., PCR Protocols: A Guide to Methods and Applications, Academic Press: San Diego, 1990.

Primers are selected to have low self- or cross-complementarity, particularly at the 3′ ends of the sequence. Long homopolymer tracts and high GC content are avoided to reduce spurious primer extension. Primers are typically about 20 residues in length, but this length can be modified as well-known in the art, in view of the particular sequence to be amplified. Computer programs are available to aid in these aspects of the design. One widely used computer program for designing PCR primers is (OLIGO 4.0 by National Biosciences, Inc., 3650 Annapolis Lane, Plymouth, Mich.). Another is Primer (Version 0.5, 1991, Whitehead Institute for Biomedical Research, Cambridge, Mass.).

Two representative primers useful for the amplification of the coding sequence of SOCS-8 are designated PCR1 and PCR2 whose sequences are disclosed below.

| | | |
|---|---|---|
| PCR1 | 5′ATGGCAGAAAATAATGAA3′ | SEQ ID NO:4 |
| PCR2 | 5′CTAGCATTGCTGTTCTGG3′ | SEQ ID NO:5 |

Exemplary PCR conditions are outlined below:

PCR Conditions

1 min @ 94° C.

30 sec @ 94° C., 4 min @ 72° C. for 5 cycles

30 sec @ 94° C., 4 min @ 70° C. for 5 cycles

30 sec @ 94° C., 4 min @ 68° C. for 25 cycles

10 min extension @ 72° C.

Specific amplification products are detected by agarose gel analysis. The contents from the PCR reactions were loaded onto a 1.2% agarose gel and electrophoresed. The DNA band of expected size are excised from the gel, placed in GenElute Agarose spin column (Supelco) and spun for 10 minutes at maximum speed in a Savant microcentrifuge. The eluted DNA was ethanol-precipitated and resuspended in 6 μl H2O for ligation.

The isolated PCR fragment containing the SOCS-8 coding sequences are ligated into a commercial vector using Invitrogen's Original TA Cloning Kit. The ligation reaction, which consists of 6 μl DNA, 1 μl 10× ligation buffer, 2 μl of plasmid pCR2.1 (25 ng/μl), Invitrogen), and 1 μl T4 DNA ligase (Invitrogen), is incubated overnight at 14° C. The reaction is heated at 65° C. for 10 minutes to inactivate the ligase enzyme, and then one microliter of the ligation reaction is transformed in One Shot cells (Invitrogen) and plated onto ampicillin plates. A single colony containing an insert is used to inoculate a 5 ml culture of LB medium. The culture is grown for 18 hours, and plasmid DNA from the culture is isolated using a Concert Rapid Plasmid Miniprep System (GibcoBRL) and sequenced to confirm that the plasmid contains the SOCS-8 insert.

Upon confirmation of the insert, the same transformant is used to inoculate a 50 ml culture of LB medium. The culture is grown for 16 hours at 37° C., and centrifuged into a cell pellet. Plasmid DNA is isolated from the pellet using a Qiagen Plasmid Midi Kit and again sequenced to confirm successful cloning of the SOCS-8 insert, using an ABI377 fluorescence-based sequencer (Perkin Elmer/Applied Biosystems Division, PE/ABD, Foster City, Calif.) and the ABI PRISM™ Ready Dye-Deoxy Terminator kit with Taq FSTM polymerase.

Each ABI cycle sequencing reaction contains about 0.5 μg of plasmid DNA. Cycle-sequencing is performed using an initial denaturation at 98° C. for 1 minute, followed by 50 cycles: 98° C. denaturation for 30 seconds, annealing at 50° C. for 30 seconds, and extension at 60° C. for 4 minutes. Temperature cycles and times are controlled by a Perkin-Elmer 9600 thermocycler. Extension products are isolated using Centriflex™ gel filtration cartridges (Advanced Genetic Technologies Corp., Gaithersburg, Md.). Each reaction product is loaded by pipette onto the column, which is then centrifuged in a swinging bucket centrifuge (Sorvall model RT6000B table top centrifuge) at 1500×g for 4 minutes at room temperature. Column-purified samples are dried under a vacuum for about 40 minutes and then dissolved in 5 μl of a DNA loading solution (83% deionized formamide, 8.3 mM EDTA, and 1.6 mg/ml Blue Dextran). The samples are then heated to 90° C. for three minutes and loaded into the gel sample wells for sequence analysis by the ABI377 sequencer. Sequence analysis is done by importing ABI377 files into the Sequencher® program (Gene Codes, Ann Arbor, Mich.). Generally sequence reads of 700 bp were obtained. Potential sequence errors are minimized by obtaining sequence information from both DNA strands and by re-sequencing difficult areas using primers at different locations until all sequencing ambiguities are removed.

It should be recognized that this method of obtaining the sequence of SEQ ID NO: 1 is exemplary and that by disclosing SEQ ID NO: 1 it provides one skilled in the art a multitude of methods of obtaining the entire sequence of SEQ ID NO: 1. By way of example, it would be possible to generate probes from the sequence disclosed in SEQ ID NO: 1 and screen human cDNA or genomic libraries and thereby obtain the entire SEQ ID NO: 1 or its genomic equivalent. Sambrook, et al., (Eds.), Molecular Cloning: A Laboratory Manual, Cold Spring Harbor Laboratory Press: Cold Spring Harbor, N.Y. (1989). Also by way of example, one skilled in the art would immediately recognize that given the sequence disclosed in SEQ ID NO: 1 it is then possible to generate the appropriate primers for PCR amplification to obtain the entire sequence represented by SEQ ID NO: 1. (see e.g., PCR Technology, H. A. Erlich, ed., Stockton Press, New York, 1989; PCR Protocols: A Guide to Methods and Applications, M. A. Innis, David H. Gelfand, John J. Sninsky, and Thomas J. White, eds., Academic Press, Inc., New York, 1990,

Host Cells and Vectors of the Invention

Autonomously replicating recombinant expression constructs such as plasmid and viral DNA vectors incorporating polynucleotides of the invention are also provided. Expression constructs wherein SOCS-8-encoding polynucleotides are operatively linked to an endogenous or exogenous expression control DNA sequence and a transcription terminator are also provided. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and preferably promote, homologous recombination in a host cell. Constructs of the invention also include sequences necessary for replication in a host cell.

Expression constructs are preferably utilized for production of an encoded protein, but also may be utilized simply to amplify a SOCS-8-encoding polynucleotide sequence.

According to another aspect of the invention, host cells are provided, including prokaryotic and eukaryotic cells, comprising a polynucleotide of the invention (or vector of the invention) in a manner which permits expression of the encoded SOCS-8 polypeptide. Polynucleotides of the invention may be introduced into the host cell as part of a circular plasmid, or as linear DNA comprising an isolated protein coding region or a viral vector. Methods for introducing DNA into the host cell well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, and mammalian cells systems.

Suitable host cells for expression of human SOCS polypeptides include prokaryotes, yeast, and higher eukaryotic cells. Suitable prokaryotic hosts to be used for the expression of human SOCS include but are not limited to bacteria of the genera Escherichia, Bacillus, and Salmonella, as well as members of the genera Pseudomonas, Streptomyces, and Staphylococcus.

The isolated nucleic acid molecules of the invention are preferably cloned into a vector designed for expression in eukaryotic cells, rather than into a vector designed for expression in prokaryotic cells. Eukaryotic cells are sometimes preferred for expression of genes obtained from higher eukaryotes because the signals for synthesis, processing, and secretion of these proteins are usually recognized, whereas this is often not true for prokaryotic hosts (Ausubel, et al., ed., in Short Protocols in Molecular Biology, 2nd edition, John Wiley & Sons, publishers, pg. 16-49, 1992.). In the case of the human SOCS-8, there are 2 consensus sequences for N-linked glycosylation, and other sites of post-translational modification can be predicted for protein kinase C phosphorylation and O-glycosylation. Eukaryotic hosts may include, but are not limited to, the following: insect cells, African green monkey kidney cells (COS cells), Chinese hamster ovary cells (CHO cells), human 293 cells, and murine 3T3 fibroblasts.

Expression vectors for use in prokaryotic hosts generally comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors (Promega), pPROEX vectors (LTI, Bethesda, Md.), Bluescript vectors (Stratagene), and pQE vectors (Qiagen).

Human SOCS-8 polypeptides may also be expressed in yeast host cells from genera including Saccharomyces, Pichia, and Kluveromyces. Yeast hosts include S. cerevisiae and P. pastoris. Yeast vectors will often contain an origin of replication sequence from a 2 micron yeast plasmid, an autonomously replicating sequence (ARS), a promoter region, sequences for polyadenylation, sequences for transcription termination, and a selectable marker gene. Vectors replicable in both yeast and E. coli (termed shuttle vectors) may also be used. In addition to the above-mentioned features of yeast vectors, a shuttle vector will also include sequences for replication and selection in E. coli. Direct secretion of human SOCS-8 polypeptides expressed in yeast hosts may be accomplished by the inclusion of nucleotide sequence encoding the yeast factor leader sequence at the 5′ end of the human SOCS-8-encoding nucleotide sequence.

Insect host cell culture systems may also be used for the expression of human SOCS-8 polypeptides. In another embodiment, the human SOCS-8 polypeptides of the invention are expressed using a baculovirus expression system. Further information regarding the use of baculovirus systems for the expression of heterologous proteins in insect cells are reviewed by Luckow and Summers, Bio/Technology 6:47 (1988).

In another embodiment, the SOCS-8 polypeptide is expressed in mammalian host cells. Non-limiting examples of suitable mammalian cell lines include the COS-7 line of monkey kidney cells (Gluzman et al., Cell 23:175 (1981)), Chinese hamster ovary (CHO) cells, and human 293 cells.

The choice of a suitable expression vector for expression of the SOCS-8 polypeptides of the invention will of course depend upon the specific host cell to be used, and is within the skill of the ordinary artisan. Examples of suitable expression vectors include pcDNA3 (Invitrogen) and pSVL (Pharmacia Biotech). Expression vectors for use in mammalian host cells may include transcriptional and translational control sequences derived from viral genomes. Commonly used promoter sequences and enhancer sequences which may be used in the present invention include, but are not limited to, those derived from human cytomegalovirus (CMV), Adenovirus 2, Polyoma virus, and Simian virus 40 (SV40). Methods for the construction of mammalian expression vectors are disclosed, for example, in Okayama and Berg (Mol. Cell. Biol. 3:280 (1983)); Cosman et al. (Mol. Immunol. 23:935 (1986)); Cosman et al. (Nature 312:768 (1984)); EP-A-0367566; and WO 91/18982.

### EXAMPLE 2

### Expression of SOCS-8 in Eukaryotic Host Cells

To produce SOCS-8 polypeptide, a SOCS-8-encoding polynucleotide is expressed in a suitable host cell using a suitable expression vector, using standard genetic engineering techniques. For example, the SOCS-8-encoding sequences described in Example 1 are subcloned into the commercial expression vector pzeoSV2 (Invitrogen, San Diego, Calif.) and transfected into Chinese Hamster Ovary (CHO) cells using the transfection reagent fuGENE 6 (Boehringer-Mannheim) and the transfection protocol provided in the product insert. Other eukcryotic cell lines, including human embryonic kidney HEK 293 and COS cells, are suitable as well. Cells stably expressing SOCS-8 are selected by growth in the presence of 100 μg/ml zeocin (Stratagene, LaJolla, Calif.). Optionally, the SOCS-8 polypeptide is isolated from the cells using standard chromatographic techniques. To facilitate purification, antisera is raised against one or more synthetic peptide sequences that correspond to portions of the SOCS-8 amino acid sequence, and the antisera is used to affinity purify SOCS-8. The SOCS-8 also may be expressed in frame with a tag sequence (e.g., polyhistidine, hemaggluttinin, FLAG) to facilitate purification. Moreover, it will be appreciated that many of the uses for SOCS-8 polypeptides, such as assays described below, do not require purification of SOCS-8 from the host cell.

Host cells of the invention are a valuable source of immunogen for development of antibodies specifically immunoreactive with SOCS-8 polypeptides. Host cells of the invention are also useful in methods for large scale production of SOCS-8 polypeptides wherein the cells are grown in a suitable culture medium and the desired polypeptide products are isolated from the cells or from the medium in which the cells are grown by purification methods known in the art, e.g., conventional chromatographic methods including immunoaffinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those wherein the desired protein is expressed and isolated as a fusion protein having a specific tag, label, or chelating moiety that is recognized by a specific binding partner or agent. The isolated protein can be cleaved to yield the desired protein, or be left as an intact fusion protein. Cleavage of the fusion component may produce a form of the desired protein having additional amino acid residues as a result of the cleavage process.

Knowledge of SOCS-8 polynucleotide sequences allows for modification of cells to permit, or increase, expression of endogenous SOCS-8 polypeptides. Cells can be modified (e.g., by homologous recombination) to provide increased expression by replacing, in whole or in part, the naturally occurring SOCS-8 promoter with all or part of a heterologous promoter so that the cells express SOCS-8 at higher levels. The heterologous promoter is inserted in such a manner that it is operatively linked to endogenous SOCS-8 encoding sequences. [See, for example, PCT International Publication No. WO 94/12650, PCT International Publication No. WO 92/20808, and PCT International Publication No. WO 91/09955.] It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the SOCS-8 coding sequence, amplification of the marker DNA by standard selection methods results in co-amplification of the SOCS-8 coding sequences in the cells.

The polynucleotide sequence information provided by the present invention also makes possible the development through, e.g. homologous recombination or “knock-out” strategies [Capecchi, Science 244:1288-1292 (1989)], of animals that fail to express functional SOCS-8 or that express a variant of a SOCS-8 polypeptide. Such animals (especially small laboratory animals such as rats, rabbits, and mice) are useful as models for studying the in vivo activities of SOCS-8 and modulators of SOCS-8.

Also made available by the invention are anti-sense polynucleotides which recognize and hybridize to polynucleotides encoding SOCS-8. Full length and fragment anti-sense polynucleotides are provided. Fragment anti-sense molecules of the invention include (i) those which specifically recognize and hybridize to SOCS-8 polynucleotdics (as determined by sequence comparison of DNA encoding SOCS-8 polypeptides to DNA encoding other known molecules). Identification of sequences unique to SOCS-8-encoding polynucleotides, can be deduced through use of any publicly available sequence database, and/or through use of commercially available sequence comparison programs. The uniqueness of selected sequences in an entire genome can be further verified by hybridization analyses. After identification of the desired sequences, isolation through restriction digestion or amplification using any of the various polymerase chain reaction techniques well known in the art can be performed. Anti-sense polynucleotides are particularly relevant to regulating expression of SOCS-8 by those cells expressing SOCS-8 mRNA.

Antisense nucleic acids (preferably 10 to 20 base pair oligonucleotides) capable of specifically binding to SOCS-8 expression control sequences or SOCS-8 RNA are introduced into cells (e.g., by a viral vector or colloidal dispersion system such as a liposome). The antisense nucleic acid binds to the SOCS-8 target nucleotide sequence in the cell and prevents transcription or translation of the target sequence. Phosphorothioate and methylphosphonate antisense oligonucleotides are specifically contemplated for therapeutic use by the invention. The antisense oligonucleotides may be further modified by poly-L-lysine, transferrin polylysine, or cholesterol moieties at their 5′ end. Suppression of SOCS-8 expression at either the transcriptional or translational level is useful to generate cellular or animal models for diseases characterized by aberrant SOCS-8 expression or as a therapeutic modality.

The SOCS-8 sequences taught in the present invention facilitate the design of novel transcription factors for modulating SOCS-8 expression in native cells and animals, and cells transformed or transfected with SOCS-8 polynucleotides. For example, the Cys2-His2 zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular SOCS-8 target sequence of the present invention facilitates the engineering of zinc finger proteins specific for the target sequence using known methods such as a combination of structure-based modeling and screening of phage display libraries [Segal et al., (1999) Proc Natl Acad Sci USA 96:2758-2763; Liu et al., (1997) Proc Natl Acad Sci USA 94:5525-30; Greisman and Pabo (1997) Science 275:657-61; Choo et al., (1997) J Mol Biol 273:525-32]. Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence [Segal et al., (1999) Proc Natl Acad Sci USA 96:2758-2763]. The artificial zinc finger repeats, designed based on SOCS-8 sequences, are fused to activation or repression domains to promote or suppress SOCS-8 expression [Liu et al., (1997) Proc Natl Acad Sci USA 94:5525-30]. Alternatively, the zinc finger domains can be fused to the TATA box-binding factor (TBP) with varying lengths of linker region between the zinc finger peptide and the TBP to create either transcriptional activators or repressors [Kim et al., (1997) Proc Natl Acad Sci USA 94:3616-3620]. Such proteins, and polynucleotides that encode them, have utility for modulating SOCS-8 expression in vivo in both native cells, animals and humans; and/or cells transfected with SOCS-8-encoding sequences. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods [McColl et al., (1999) Proc Natl Acad Sci USA 96:9521-6; Wu et al., (1995) Proc Natl Acad Sci USA 92:344-348]. The present invention contemplates methods of designing such transcription factors based on the gene sequence of the invention, as well as customized zinc finger proteins, that are useful to modulate SOCS-8 expression in cells (native or transformed) whose genetic complement includes these sequences.

### Polypeptides of the Invention

The invention also provides isolated mammalian SOCS-8 polypeptides encoded by a polynucleotide of the invention. A human SOCS-8 polypeptide amino acid sequence is set out in SEQ ID NO: 2. The invention also embraces polypeptides that have at least 99%, at least 95%, at least 90%, at least 85%, at least 80%, at least 75%, at least 70%, at least 65%, at least 60%, at least 55% or at least 50% identity and/or homology to the polypeptide polypeptide set out in SEQ ID NO: 2 with the proviso that such a homologous polypeptide retains the ability to interact with its cognate binding partner polypeptide.

Percent amino acid sequence “identity” with respect to the polypeptide of SEQ ID NO: 2 is defined herein as the percentage of amino acid residues in the candidate sequence that are identical with the residues in the SOCS-8 sequence after aligning both sequences and introducing gaps, if necessary, to achieve the maximum percent sequence identity, and not considering any conservative substitutions as part of the sequence identity. Percent sequence “homology” with respect to the polypeptide of SEQ ID NO: 2 is defined herein as the percentage of amino acid residues in the candidate sequence that are identical with the residues in the SOCS-8 sequence after aligning the sequences and introducing gaps, if necessary, to achieve the maximum percent sequence identity, and also considering any conservative substitutions as part of the sequence identity.

In one aspect, percent homology is calculated as the percentage of amino acid residues in the smaller of two sequences which align with identical amino acid residue in the sequence being compared, when four gaps in a length of 100 amino acids may be introduced to maximize alignment [Dayhoff, in Atlas of Protein Sequence and Structure, Vol. 5, p. 124, National Biochemical Research Foundation, Washington, D.C. (1972), incorporated herein by reference].

Polypeptides of the invention may be isolated from natural cell sources or may be chemically synthesized, but are preferably produced by recombinant procedures involving host cells of the invention. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated form of SOCS-8 polypeptides are embraced.

Overexpression in eukaryotic and prokaryotic hosts as described above facilitates the isolation of SOCS-8 polypeptides. The invention therefore includes isolated SOCS-8 polypeptides as set out in SEQ ID NO:2 and variants and conservative amino acid substitutions therein including labeled and tagged polypeptides.

The invention includes SOCS-8 polypeptides which are “labeled”. The term “labeled” is used herein to refer to the conjugating or covalent bonding of any suitable detectable group, including enzymes (e.g., horseradish peroxidase, beta-glucuronidase, alkaline phosphatase, and beta-D-galactosidase), fluorescent labels (e.g., fluorescein, luciferase), and radiolabels (e.g., ¹⁴C, ¹²⁵I, ³H, ³²P, and ³⁵S) to the compound being labeled. Techniques for labeling various compounds, including proteins, peptides, and antibodies, are well known. See, e.g., Morrison, Methods in Enzymology 32b, 103 (1974); Syvanen et al., J. Biol. Chem. 284, 3762 (1973); Bolton and Hunter, Biochem. J. 133, 529 (1973). The termed labelled may also encompass a polypeptide which has covalently attached an amino acid tag as discussed below.

In addition, the SOCS-8 polypeptides of the invention may be indirectly labeled. This involves the covalent addition of a moiety to the polypeptide and subsequent coupling of the added moiety to a label or labeled compound which exhibits specific binding to the added moiety. Possibilities for indirect labeling include biotinylation of the peptide followed by binding to avidin coupled to one of the above label groups. Another example would be incubating a radiolabeled antibody specific for a histidine tag with a SOCS-8 polypeptide comprising a polyhistidine tag. The net effect is to bind the radioactive antibody to the polypeptide because of the considerable affinity of the antibody for the tag.

The invention also embraces variants (or analogs) of the SOCS-8 protein. In one example, insertion variants are provided wherein one or more amino acid residues supplement a SOCS-8 amino acid sequence. Insertions may be located at either or both termini of the protein, or may be positioned within internal regions of the SOCS-8 protein amino acid sequence. Insertional variants with additional residues at either or both termini can include for example, fusion proteins and proteins including amino acid tags or labels. Insertion variants include SOCS-8 polypeptides wherein one or more amino acid residues are added to a SOCS-8 acid sequence, or to a biologically active fragment thereof.

Insertional variants therfore can also include fusion proteins wherein the amino and/or carboxy termini of SOCS-8 is fused to another polypeptide. Various tag polypeptides and their respective antibodies are well known in the art. Examples include poly-histidine (poly-his) or poly-histidine-glycine (poly-his-gly) tags; the influenza HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an alpha-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397(1990)]. In addition, the SOCS-8 polypeptide can be tagged with enzymatic proteins such as peroxidase and alkaline phosphatase.

In another aspect, the invention provides deletion variants wherein one or more amino acid residues in a SOCS-8 polypeptide are removed. Deletions can be effected at one or both termini of the SOCS-8 polypeptide, or with removal of one or more residues within the SOCS-8 amino acid sequence. Deletion variants, therefore, include all fragments of the SOCS-8 polypeptide.

The invention also embraces polypeptide fragments of the sequence set out in SEQ ID NO: 2 wherein the fragments maintain biological (e.g., ligand binding or RNA binding and/or other biological activity) Fragments comprising at least 5, 10, 15, 20, 25, 30, 35, or 40 consecutive amino acids of SEQ ID NO: 2 are comprehended by the invention. Fragments of the invention having the desired biological properties can be prepared by any of the methods well known and routinely practiced in the art.

The present invention also includes include variants of the aforementioned polypeptide, that is polypeptides that vary from the referents by conservative amino acid substitutions, whereby a residue is substituted by another with like characteristics. Variant polypeptides include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table 2 (from WO 97/09433, page 10, published Mar. 13, 1997 (PCT/GB96/02197, filed Sep. 6, 1996), immediately below.

TABLE 2
Conservative Substitutions I
SIDE CHAIN

| CHARACTERISTIC | AMINO ACID |
|---|---|
| Aliphatic | |
| Non-polar | G A P |
| | I L V |
| Polar - uncharged | C S T M |
| | N Q |
| Polar - charged | D E |
| | K R |
| Aromatic | H F W Y |
| Other | N Q D E |

Alternatively, conservative amino acids can be grouped as described in Lehninger, [Biochemistry, Second Edition; Worth Publishers, Inc. NY:N.Y. (1975), pp.71-77] as set out in Table 3, immediately below

TABLE 3
Conservative Substitutions II
SIDE CHAIN

| CHARACTERISTIC | AMINO ACID |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

As still an another alternative, exemplary conservative substitutions are set out in Table 4, immediately below.

TABLE 4
Conservative Substitutions III

| Original | Exemplary |
|---|---|
| Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

Generally it is anticipated that the SOCS-8 polypeptide will be found primarily intracellularly, the intracellular material can be extracted from the host cell using any standard technique known to the skilled artisan. For example, the host cells can be lysed to release the contents of the cytoplasm by homogenization, and/or sonication followed by centrifugation. The SOCS-8 polypeptide is found primarily in the supernatant after centrifugation of the cell homogenate, and the SOCS-8 polypeptide can be isolated by way of non-limiting example by any of the methods below.

In those situations where it is preferable to partially or completely isolate the SOCS-8 polypeptide, purification can be accomplished using standard methods well known to the skilled artisan. Such methods include, without limitation, separation by electrophoresis followed by electroelution, various types of chromatography (immunoaffinity, molecular sieve, and/or ion exchange), and/or high pressure liquid chromatography. In some cases, it may be preferable to use more than one of these methods for complete purification.

Purification of SOCS-8 polypeptide can be accomplished using a variety of techniques. If the polypeptide has been synthesized such that it contains a tag such as Hexahistidine (SOCS-8/hexaHis) or other small peptide such as FLAG (Eastman Kodak Co., New Haven, Conn.) or myc (Invitrogen, Carlsbad, Calif.) at either its carboxyl or amino terminus, it may essentially be purified in a one-step process by passing the solution through an affinity column where the column matrix has a high affinity for the tag or for the polypeptide directly (i.e., a monoclonal antibody specifically recognizing SOCS-8). For example, polyhistidine binds with great affinity and specificity to nickel, thus an affinity column of nickel (such as the Qiagen Registered ™ nickel columns) can be used for purification of SOCS-8/polyHis. (See for example, Ausubel et al., eds., Current Protocols in Molecular Biology, Section 10.11.8, John Wiley & Sons, New York [1993]).

Even if the SOCS-8 polypeptide is prepared without a label or tag to facilitate purification. The SOCS-8 of the invention may be purified by immunoaffinity chromatography. To accomplish this, antibodies specific for the SOCS-8 polypeptide must be prepared by means well known in the art. Antibodies generated against the SOCS-8 polypeptides of the invention can be obtained by administering the polypeptides or epitope-bearing fragments, analogues or cells to an animal, preferably a nonhuman, using routine protocols. For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in Monoclonal Antibodies and Cancer Therapy, Alan R. Liss, Inc. (1985).

Where the SOCS-8 polypeptide is prepared without a tag attached, and no antibodies are available, other well known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity. A representative purification scheme is detailed below.

Variants that display inhibitory properties of native SOCS-8 and are expressed at higher levels and variants that provide for constitutive active SOCS-8 polypeptide are particularly useful in assays of the invention and also useful in cellular and animal models for diseases characterized by aberrant SOCS-8 expression activity.

It should be understood that the definition of SOCS-8 polypeptides of the invention is intended to include polypeptides bearing all the modifications herein described with the proviso that such modified SOC-8 polypeptides retain the ability to interact with a binding partner polypeptide.

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for SOCS-8 or fragments thereof. Antibodies of the invention include human antibodies which are produced and identified according to methods described in WO93/11236, published Jun. 20, 1993, which is incorporated herein by reference in its entirety. Antibody fragments, including Fab, Fab′, F(ab)₂, and Fv, are also provided by the invention. The term “specific for,” when used to describe antibodies of the invention, indicates that the variable regions of the antibodies of the invention recognize and bind SOCS-8 polypeptides exclusively (i.e., able to distinguish SOCS-8 polypeptides from other known polypeptides by virtue of measurable differences in binding affinity, despite the possible existence of localized sequence identity, homology, or similarity between SOCS-8 and such polypeptides). It will be understood that specific antibodies may also interact with other proteins (for example, S. aureus protein A or other antibodies in ELISA techniques) through interactions with scquences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the SOCS-8 polypeptides of the invention are also contemplated, provided that the antibodies are, first and foremost, specific for SOCS-8 polypeptides. Antibodies of the invention can be produced using any method well known and routinely practiced in the art.

Non-human antibodies may be humanized by any methods known in the art. In one method, the non-human CDRs are inserted into a human antibody or consensus antibody framework sequence. Further changes can then be introduced into the antibody framework to modulate affinity or immunogenicity.

Antibodies of the invention are useful for, diagnostic purposes to detect or quantitate SOCS-8, as well as purification of SOCS-8. Kits comprising an antibody of the invention for any of the purposes described herein are also comprehended. In general, a kit of the invention also includes a control antigen for which the antibody is immunospecific

### EXAMPLE 3

### Generating Antibodies to SOCS-8

Standard techniques are employed to generate polyclonal or monoclonal antibodies to the SOCS-8 enzyme, and to generate useful antigen-binding fragments thereof or variants thereof, including “humanized” variants. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988); and other documents cited below. In one embodiment, recombinant SOCS-8 polypeptides (or cells or cell membranes containing such polypeptides) are used as antigen to generate the antibodies. In another embodiment, one or more peptides having amino acid sequences corresponding to an immunogenic portion of a SOCS-8 polypeptide (e.g., 6, 7, 8, 9, 10, 11, 12, 13, 14, 15, 16, 17, 18, 19, 20, or more amino acids) are used as antigen

In order to mimic a protein epitope with a small synthetic peptide, it is important to choose a sequence that is hydrophilic, surface-oriented, and flexible. Van Regenmortel, 1986. This is because most naturally occurring proteins found in physiological solutions have their hydrophilic residues on the surface and their hydrophobic residues buried. Antibodies generally bind to epitopes on the surfaces of naturally occurring proteins. Several known epitopes have a high degree of mobility The N- and C-termini of proteins are generally surface-oriented since they contain charged groups, i.e., NH₃⁺ and COO⁻. They often have a high degree of mobility as well, since they are located at the ends. These termini are often chosen as candidates for synthesis because they possess all three properties. Peptides corresponding to surface residues of SOCS-8, especially hydrophilic portions are contemplated. Also contemplated are peptides located at the amino and carboxy terminal ends of SOCS-8

One skilled in the art recognizes that algorithms have been developed to assign values of hydrophilicity, surface accessibility, and flexibility to each amino acid residue within a given protein sequence. The same has been done to assign an antigenic index to each residue, giving an indication of how antigenic that residue is within a specific sequence. Hopp and Woods, Mol. Immunol, 1983 20(4): p. 483-9, Hopp and Woods, Proc. Natl Acad. Sci USA 1981, 78(6) p. 3824-8. Although selection of hydrophilic segments has been widely used in generating anti-peptide antibodies that are useful for binding native antigen. Unlike antibodies however, T cell receptors see relatively small segments of protein antigen after cleavage and unfolding. T cell antigenic sites has also been addressed by predictive computer models. Margalit, H. et al., J. Immunol. 1987 138(7): pg 2213-29 Computer programs useful for the prediction of epitopes are commercially available. For example MacVector® (Oxford Molecular, Oxford, UK) and Protean® (DNAStar Madison, Wis. 53715) Once a peptide antigen is selected and synthesized the antigen may be mixed with an adjuvant or linked to a hapten to increase antibody production.

A. Polyclonal or Monoclonal Antibodies

As one exemplary protocol, recombinant SOCS-8 or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanine (Pierce), according to the manufacturer's recommendations. For an initial injection, the antigen is emulsified with Freund's Complete Adjuvant and injected subcutaneously. At intervals of two to three weeks, additional aliquots of SOCS-8 antigen are emulsified with Freund's Incomplete Adjuvant and injected subcutaneously. Prior to the final booster injection, a serum sample is taken from the immunized mice and assayed by western blot to confirm the presence of antibodies that immunoreact with SOCS-8. Serum from the immunized animals may be used as a polyclonal antisera or used to isolate polyclonal antibodies that recognize SOCS-8. Alternatively, the mice are sacrificed and their spleen removed for generation of monoclonal antibodies.

To generate monoclonal antibodies, the spleens are placed in 10 ml serum-free RPMI 1640, and single cell suspensions are formed by grinding the spleens in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75 mM Hepes, pH 8.0) (Boehringer Mannheim) is stirred into the pellet, followed by the addition of scrum-free RPMI. Thereafter, the cells are centrifuged and resuspended in RPMI containing 15% FBS, 100 μM sodium hypoxanthine, 0.4 μM aminopterin, 16 μM thymidine (HAT) (Gibco), 25 units/ml IL-6 (Boehringer Mannheim) and 1.5×106 thymocytes/ml and plated into 10 Corning flat-bottom 96-well tissue culture plates (Corning, Corning N.Y.).

On days 2, 4, and 6, after the fusion, 100 μl of medium is removed from the wells of the fusion plates and replaced with fresh medium. On day 8, the fusions are screened by ELISA, testing for the presence of mouse IgG that binds to SOCS-8. Selected fusion wells are further cloned by dilution until monoclonal cultures producing anti-SOCS-8 antibodies are obtained.

C. Human SOCS-8-Neutralizing Antibodies from Phage Display

Human SOCS-8-neutralizing antibodies are generated by phage display techniques such as those described in Aujame et al., Human Antibodies, 8(4):155-168 (1997); Hoogenboom, TIBTECH; 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is panned (screened) for SOCS-8-specific phage-antibodies using labelled or immobilized SOCS-8 as antigen-probe.

D. Human SOCS-8-Neutralizing Antibodies From Transgenic Mice

Human SOCS-8-neutralizing antibodies are generated in transgenic mice essentially as described in Bruggemann and Neuberger, Immunol. Today, 17(8):391-97 (1996) and Bruggemann and Taussig, Curr. Opin. Biotechnol., 8:455-58 (1997). Transgenic mice carrying human V-gene segments in germline configuration and that express these transgenes in their lymphoid tissue are immunized with a SOCS-8 composition using conventional immunization protocols. Hybridomas are generated using B cells from the immunized mice using conventional protocols and screened to identify hybridomas secreting anti-SOCS-8 human antibodies (e.g., as described above).

### Assays of the Invention

The invention includes several assay systems for identifying SOCS-8 modulator compounds.

Each cytokine which utilizes the JAK-STAT signal transduction pathway activates a distinct combination of members of the JAK and STAT families. Thus, either the JAKs, the STATs or both could contribute to the specificity of ligand action. The JAK-STAT signal transduction pathway was first discovered for interferon alpha (IFN-alpha) and interferon gamma (IFN-gamma) by the complementation of mutant cell lines defective in response to IFN-gamma and/or IFN-alpha [Velazquez et al., Cell, 70:313-322 (1992); Watling et al., Nature, 366:230-235 (1993); Muller et al., Nature, 366:129-135 (1993) and EMBO, 12:4221-4228 (1993); Darnell et al., Science, 264:1415-1421 (1994); Leung et al., Mol. Cell. Biol., 15:1312-1317 (1995)]. It has subsequently been shown that the same general pathway is activated by most cytokines and some growth factors [for review, see Ihle and Kerr, Trends Genet., 11:69-74 (1995); Taniguchi, Science, 268:251-255 (1995)]. This pathway is activated predominantly through receptors which do not possess intrinsic intracellular kinase domains and belong to the class I or class II cytokine receptor superfamily. The lack of inherent catalytic activity in these receptors is overcome through the use of receptor-associated kinases of the Janus kinase (JAK) family. Upon ligand binding, the receptor chains oligomerize allowing the associated kinases to interact and likely crossactivate each other by tyrosine phosphorylation. Subsequently, the activated JAKs directly phosphorylate the intracellular domains of the receptors on specific tyrosine residues. This phosphorylationm allows the selective recruitment of SH2-domain containing proteins, particularly STATs (Signal transducers and activators of transcription), through a specific interaction between the STAT SH2 domains and the phosphotyrosines within the STAT recruitment sites of the intracellular domains of the receptor chains. These receptor-associated STATs are then rapidly phosphorylated, likely by the activated JAKs[Quelle et al., supra]. The phosphorylation of the STATs is followed by STAT dimerization, translocation to the nucleus and activation of cytokine inducible genes.

The JAK and STAT families are growing rapidly. The JAK family consists of four members so far: JAK1, JAK2, JAK3 and Tyk2 [Wilks et al., Mol. Cell. Biol., 11:2057-2065 (1991); Silvennoinen et al., Proc. Natl. Acad. Sci. USA, 90:8429-8433 (1993); Firmbach-Kraft et al., Oncogene, 5:1329-1336 (1990); Witthuhn et al., Cell, 742:27-236 (1994); Kawamuraet al., Proc. Natl. Acad. Sci. USA, 91:6374-6378 (1994); for review see Ziemiecki et al., Trends Cell Biol., 4:207-212 (1994); Ihle et al., Trends in Biochemical Sciences, 19:222-227 (1994); Ihle et al., Trends Genet., 11:69-74 (1995); Ihle and Kerr, supra]. The STAT family now includes seven different members, which have been cloned: Stat 1 alpha, Stat 1 beta, and Stats2-6 [Schindler et al., Proc. Natl. Acad. Sci. USA, 89:7836-7839 (1992); Fu et al., Proc. natl. Acad. Sci. USA, 89:7840-7843 (1992); Zhong et al., Science, 264:95-98 (1994) and Proc. Natl. Acad. Sci. USA, 91:4806-4810 (1994); Yamamoto et al., Mol. Cell. Biol., 14:4342-4349 (1994); Akira et al., Cell, 77:63-71 (1994); Wakao et al., Cell, 70:2182-2191(1994); Hou et al., Science, 265:1701-1706 (1994); International Patent Publication WO 95/08629, by Darnell et al., published Mar. 30, 1995; and International Patent Publication No. WO 93/19179, by Darnell et al., published Sep. 30, 1993] and several others, which were identified by electrophoretic mobility shift assays, but have not been cloned yet [Meyer et al., J. Biol. Chem., 269:4701-4704 (1994); Finbloom et al., Mol. Cell. Biol., 14:2113-2118 (1994); Tian et al., Blood, 84:1760-1764 (1994); Finbloom and Winestock, J. Immunol., 155:1079-1090 (1995); Frank et al., Proc. Natl. Acad. Sci. USA, 92:7779-7783 (1995)].

The JAK kinases are characterized by seven conserved domains: two PTK-related domains and five domains with unknown functions [Ziemiecki et al., supra]. The main difference between JAKs and other protein tyrosine kinases (PTK) is that along with a kinase domain, shown to be active [Wilks et al., supra], they also contain a PTK-like domain with substitutions of several residues essential for kinase activity. Thus, the second domain is expected to be inactive as a PTK and probably has some other function. Another feature of this family is the lack of any detectable SH2 or SH3 domains. The functions of the other five regions of homology are also unknown.

STATs represent proteins containing SH2, SH3 and DNA-binding domains [for reviews, see Darnell et al., supra; Fu, Journal of Leukocyte Biology, 57:529-535 (1995)]. The highly selective and specific interaction between Stat SH2 domains and the phosphotyrosine containing Stat recruitment sites on the intracellular domains of the cytokine receptors determines which Stats are to be recruited to a particular receptor complex [Heim et al., Science, 267:1347-1349 (1995); Stahl et al., Science, 267:1349-1353 (1995)].

The intracellular domain of each cytokine receptor specifically associates with one or more distinct JAKs. While some JAKs and STATs participate in several cytokine signalling pathways, others are more restricted. In the case of JAKs, Jak2 and, especially Jak1, associate with receptors participating in different apparently unrelated cytokine-receptor systems [for reviews, see Ziemiecki et al., supra; Ihle et al., supra; Taniguchi, supra; Ihle and Kerr, supra]. Jak3 appears to be restricted to the ligand receptor systems through its association with the IL-2R gamma [c]chain [Johnston et al., Nature, 370:151-153 (1994); Witthuhn et al., supra; Russell et al., Science, 266:1042-1045 (1994); Miyazaki et al., Science, 266:1045-1047 (1994); Tanaka et al., Proc. Natl. Acad. Sci. USA, 91:7271-7275 (1994)]. Tyk2 was shown to be activated during IFN-alpha signalling [Velazquez et al., supra; Barbieri et al., Eur. J. Biochem., 223:427435 (1994)] and also during CNTF-related cytokine signalling, albeit only in certain cell types [Stahl et al., Science, 263:92-95 (1994); Lutticken et al., Science, 262:89-92 (1994)]. Recently, the activation of Tyk2 by IL-10 and IL-12 was shown [Finbloom and Winestock, supra; Bacon et al., J. Exp. Med., 181:399404 (1995); Ho et al., Mol. Cell. Biol., 15:5043-5053 (1995)]. Thus, the Jaks may contribute to the specificity of signal transduction at some level.

Phosphorylation of cytokine receptor subunits, JAK kinases and STAT transcription factors are substantially reduced in cells expressing at least one SOCS (SOCS-1) suggesting that SOCS target an early effector in this pathway. The SOCS-8 polypeptides of the invention interact with at least some members of the JAK/STAT regulatory effector family of molecules.

SOCS-8 polypeptides bind to an activated receptor-JAK complex, either in a direct manner or via a bridging molecule. An activated receptor-JAKcomplex can be obtained in several different ways; by immunoprecipitation of membrane extracts from normal cells or tissues, or from cells that have been genetically engineered to express the receptor-JAKcomplex. Alternatively, relevant parts of the receptor and the JAK proteins can be recombinantly expressed and purified. An in vitro assay would then be designed to first establish an assay that would detect the binding between the receptor-JAK complex and the preferably recombinantly expressed SOCS-8 protein. For this purpose an easy detection system should be available for at least one of the partners in the complex; such a detection system could be based on antibodies, or on labeling one of the proteins with a marker molecule or radioactivity. The subsequent use of this assay would be to screen a compound collection for substances that would modulate the interaction between the receptor-JAK complex and SOCS-8

In solution assays, methods of the invention comprise the steps of (a) contacting a SOCS-8 polypeptide with one or more candidate inhibitor compounds and (b) identifying the compounds that modulate the SOCS-8 association with a binding partner polypeptide Agents that modulate (i.e., increase, decrease) SOCS-8 association with other proteins or expression may be identified by incubating a putative modulator with a cell expressing a SOCS-8 polypeptide or polynucleotide and determining the effect of the putative modulator on SOCS-8 activity or expression. The selectivity of a compound that modulates the activity of SOCS-8 can be evaluated by comparing its effects on SOCS-8 to its effect on other SOCS polypeptides. Modulators of SOCS-8 activity will be therapeutically useful in treatment of diseases and physiological conditions in which normal or aberrant SOCS-8 activity is involved.

The propensity of a specific protein to interact with SOCS-8 may be determined in a variety of ways. To determine this propensity for association with SOCS-8, a yeast two-hybrid screen can performed with SOCS-8 as the “bait” and a cDNA library or an embryonic cell library as the “prey”. Additional biochemical binding assays that may prove useful for identifying compounds that are able to associate with SOCS-8 are well known in the art including, but not limited to: equilibrium or membrane flow dialysis, antibody binding assays, gel-shift assays, in vitro binding assays, filter binding assays, enzyme-linked immunoabsorbent assays (ELISA), western blots, co-immunoprecipitation, immunogold co-immunoprecipitation, coimmunolocalization, co-crystallization, fluorescence energy transfer, competition binding assays, chemical crosslinking, and affinity purification. In addition, genetic analysis may be used to identify accessory proteins that interact with SOCS-8 or are peripherally involved in SOCS-8 function

### EXAMPLE 5

### Yeast Two Hybrid Cell Based Screen

The construction of yeast two-hybrid systems is generally known. This approach identifies protein-protein interactions in vivo through reconstitution of a transcriptional activator (Fields and Song (1989) Nature 340: 245), such as the yeast Gal4 transcription protein. The yeast Gal4 protein, which consists of separable domains responsible for DNA-binding and transcriptional activation. Polynucleotides encoding two hybrid proteins, one consisting of the yeast Gal4 DNA-binding domain fused to a polypeptide sequence of a known protein and the other consisting of the Gal activation domain fused to a polypeptide sequence of a second protein, are constructed and introduced into a yeast host cell. Intermolecular binding between the two fusion proteins reconstitutes the Ga14 DNA-binding domain with the Gal4 activation domain, which leads to the transcriptional activation of a reporter gene (e.g., lacZ, HIS3) which is operably linked to a Gal4 binding site. Typically, the two hybrid method is used to identify novel polypeptide sequences which interact with a known protein (Silver S C and Hunt S W (1993) Mol. Biol. Rep. 17: 155; Durfee et al. (1993) Genes Devel. 7; 555; Yang et al. (1992) Science 257: 680; Luban et al. (1993) Cell 73: 1067; Hardy et al. (1992) Genes Devel. 6; 801; Bartel et al. (1993) Biotechniques 14: 920; and Vojtek et al. (1993) Cell 74: 205). As applied to the present case, the two-hybrid system permits identification of peptide sequences which interact with SOCS-8, and therefore, are potential SOCS-8 binding partners.

The invention provides hybrid screening assays and related host organisms (typically unicellular organisms) which harbor a mammalian SOCS-8 polypeptide two-hybrid system, typically in the form of polynucleotides encoding a first hybrid protein, a second hybrid protein, and a reporter gene, wherein said polynucleotide(s) are either stably replicated or introduced for transient expression. In an embodiment, the host organism is a yeast cell (e.g., Saccharomyces cervisiae) in which the reporter gene transcriptional regulatory sequence comprises a Gal4-responsive promoter.

Yeast comprising (1) an expression cassette encoding a GAIA DNA binding domain (or GAL4 activator domain) fused to a binding fragment of SOCS-8 capable of binding to a partner protein (2) an expression cassette encoding a GALA DNA activator domain (or GAL4 binding domain, respectively) fused to a member of a cDNA library or a binding fragment of a DNA polymerase capable of binding to a mammalian DNA SOCS-8 polypeptide, and (3) a reporter gene (e.g., beta-galactosidase) comprising a cis-linked GALA transcriptional response element can be used for agent screening. Such yeast are incubated with a test agent and expression of the reporter gene (e.g., beta-galactosidase) is determined; the capacity of the agent to inhibit expression of the reporter gene as compared to a control culture identifies whether the candidate agent is a SOCS-8 modulatory agent.

Yeast two-hybrid systems may be used to screen a mammalian (typically human) cDNA expression library, wherein human cDNA is fused to a GALA DNA binding domain or activator domain, and either SOCS-8 polypeptide sequence is fused to a GALA activator domain or DNA binding domain, respectively. Such a yeast two-hybrid system can screen for cDNAs that encode proteins which bind to SOCS-8 sequences (binding partner polypeptides). For example, a cDNA library can be produced from mRNA from a embryonic or other suitable cell type. Such a cDNA library cloned in a yeast two-hybrid expression system (Chien et al. (1991) Proc. Natl. Acad. Sci. (U.S.A.) 88: 9578) can be used to identify cDNAs which encode proteins that interact with SOCS-8 and thereby produce expression of the GALA-dependent reporter gene.

Once binding partner polypeptide species are identified, in-vitro binding assays can be performed with SOCS-8 and the identified protein(s). Small molecule effectors that modify SOCS function are useful therepeutics for the treatment of certain diseases including inflammatory disorders, metabolic disorders, growth disorders and cancers.

### EXAMPLE 6

### Binding Assays For Detecting SOCS-8 Modulators

Administration of an efficacious dose of an agent capable of specifically inhibiting SOCS-8: binding partner complex formation to a patient can be used as a therapeutic or prophylactic method for treating pathological conditions (e.g., cancer, inflammation, lymphoproliferative diseases, autoimmune disease, neurodegenerative diseases, and the like) Thus, assays which monitor SOCS-8: binding partner binding are of value in screening for SOCS-8 modulators.

Binding assays often take one of two forms: immobilized SOCS-8 polypeptide(s) can be used to bind labeled binding partner polypeptide(s), or conversely, immobilized binding partner polypeptide(s) can be used to bind labeled SOCS-8 polypeptides. In each case, the labeled polypeptide is contacted with the immobilized polypeptide under conditions that permit specific binding of the polypeptides(s) to form a complex in the absence of added agent. Particular aqueous conditions may be selected by the practitioner according to conventional methods. For general guidance, the following buffered aqueous conditions may be used: 10-250 mM NaCl, 5-50 mM Tris HCl, pH 5-8, with optional addition of divalent cation(s) and/or metal chelators and/or nonionic detergents and/or membrane fractions. Additions, deletions, modifications (such as pH) and substitutions (such as KCl substituting for NaCl or buffer substitution) may be made to these basic conditions. Modifications can be made to the basic binding reaction conditions so long as specific binding of SOCS-8 polypeptide(s) to binding partner polypeptides occurs in the control reaction(s). In such reactions, at least one polypeptide species typically is labeled with a detectable marker. Suitable labeling includes, but is not limited to, radiolabeling by incorporation of a radiolabeled amino acid (e.g., ¹⁴C-labeled leucine, ³H-labeled glycine, ³⁵S-labeled methionine), radiolabeling by post-translational radioiodination with 1251 or ¹³¹I (e.g., Bolton-Hunter reaction and chloramine T), labeling by post-translational phosphorylation with ³²P (e.g., phosphorylase and inorganic radiolabeled phosphate) fluorescent labeling by incorporation of a fluorescent label (e.g., fluorescein or rhodamine), or labeling by other conventional methods known in the art. In embodiments where one of the polypeptide species is immobilized by linkage to a substrate, the other polypeptide is generally labeled with a detectable marker.

Additionally, in some embodiments a SOCS-8 or binding partner polypeptide may be used in combination with an accessory protein (e.g., a protein which forms a complex with the polypeptide in vivo). It is typically preferred that different labels are used for each polypeptide species, so that binding of individual and/or heterodimeric and/or multimeric complexes can be readily distinguished. For example but not by way of limitation, a SOCS-8 polypeptide is labeled with fluorescein and an accessory polypeptide is labeled with a fluorescent marker that fluoresces with either a different excitation wavelength or emission wavelength, or both. Alternatively, double-label scintillation counting is used, wherein a SOCS-8 polypeptide is labeled with one isotope (e.g., ³H) and a binding partner polypeptide species is labeled with a different isotope (e.g., ¹⁴C) that can be distinguished by scintillation counting using standard discrimination techniques.

Labeled polypeptide(s) are contacted with immobilized polypeptide(s) under aqueous conditions as described herein. The time and temperature of incubation of a binding reaction is optionally varied, with the selected conditions permitting specific binding to occur in a control reaction where no agent is present. Preferable embodiments employ a reaction temperature of about at least 15 degrees Centigrade, more preferably 30 to 42 degrees Centigrade, and a time of incubation of approximately at least 15 seconds, although longer incubation periods, from 30 seconds to a minute to several minutes or more, are preferable so that, in some embodiments, a binding equilibrium is attained. Binding kinetics and the thermodynamic stability of bound SOCS-8: binding partner complexes determine the latitude available for varying the time, temperature, salt, pH, and other reaction conditions. However, for any particular embodiment, desired binding reaction conditions can be calibrated readily by the practitioner using conventional methods in the art, which may include binding analysis using Scatchard analysis, Hill analysis, and other standard analytic methods (Proteins, Structures and Molecular Principles, (1984) Creighton (ed.), W. H. Freeman and Company, New York).

Specific binding of labeled SOCS-8 or binding partner polypeptide to immobilized SOCS-8 or binding partner polypeptide, respectively, is determined by including unlabeled competitor protein(s) (e.g., albumin). Similarly, specific binding of labeled SOCS-8 or binding partner polypeptide to immobilized binding partner or SOCS-8 polypeptide, respectively, is determined by including unlabeled competitor protein(s) (e.g., albumin). After a binding reaction is completed, labeled polypeptide(s) specifically bound to immobilized polypeptide is detected. For example and not by way of limitation, after a suitable incubation period for binding, the aqueous phase containing non-immobilized protein is removed and the substrate containing the immobilized polypeptide species and any labeled protein bound to it is washed with a suitable buffer, optionally containing unlabeled blocking agent(s), and the wash buffer(s) removed. After washing, the amount of detectable label remaining specifically bound to the immobilized polypeptide is determined (e.g., by optical, enzymatic, autoradiographic, or other radiochemical methods).

In some embodiments, addition of unlabeled blocking agents that inhibit non-specific binding are included. Examples of such blocking agents include, but are not limited to, the following: calf thymus DNA, salmon sperm DNA, yeast RNA, mixed sequence (random or pseudorandom sequence) oligonucleotides of various lengths, bovine serum albumin, nonionic detergents (NP-40, Tween, Triton X-100, etc.), nonfat dry milk proteins, Denhardt's reagent, polyvinylpyrrolidone, Ficoll, and other blocking agents. Practitioners may, in their discretion, select blocking agents at suitable concentrations to be included in binding assays; however, reaction conditions are selected so as to permit specific binding between a SOCS-8 polypeptide and a binding partner polypeptide in a control binding reaction. Blocking agents are included to inhibit nonspecific binding of labeled protein to immobilized protein and/or to inhibit nonspecific binding of labeled polypeptide to the immobilization substrate.

In embodiments where a polypeptide is immobilized, covalent or noncovalent linkage to a substrate may be used. Covalent linkage chemistries include, but are not limited to, well-characterized methods known in the art (Kadonaga and Tijan (1986) Proc. Natl. Acad. Sci. (U.S.A.) 83: 5889). One example, not for limitation, is covalent linkage to a substrate derivatized with cyanogen bromide (such as CNBr-derivatized Sepharose 4B). It may be desirable to use a spacer to reduce potential steric hindrance from the substrate. Noncovalent bonding of proteins to a substrate include, but are not limited to, bonding of the protein to a charged surface (e.g., on a bead) and binding with specific antibodies. In one class of embodiments, parallel binding reactions are conducted,

wherein one set of reactions serves as control and at least one other set of reactions include various quantities of agents, mixtures of agents, or biological extracts, that are being tested for the capacity to inhibit binding of a SOCS-8 polypeptide to a binding partnerpolypeptide or disrupt, modulate, inhibit, or potentiate the activity of either or both. Agents which, when added to a binding reaction, inhibit formation of SOCS-8: binding partner complexes are thereby identified as SOCS-8 inhibitors;

Agents which, when added to a binding reaction, enhance formation of SOCS-8: binding partner complexes are thereby identified as SOCS-8 potentiators (e.g., SOCS-8 agonists;)

In a preferred embodiment, several binding reactions are monitored simultaneously, e.g., using a format which permits simultaneous analysis of several samples (microtiter plates, etc.). In a preferred embodiment, the assays are automated, e.g., using robotics for pipetting samples into microtiter plates.

One means for detecting binding of a SOCS-8 polypeptide to a binding partner polypeptide is to immobilize the SOCS-8 polypeptide, such as by covalent or noncovalent chemical linkage to a solid support, and to contact the immobilized SOCS-8 polypeptide with a binding partner polypeptide that has been labeled with a detectable marker (e.g., by incorporation of radiolabeled amino acid, by epitope tagging and reporting with a fluorescent-labelled anti-epitope tag antibody, and the like). Such contacting is typically performed in aqueous conditions which permit binding of a SOCS-8 polypeptide to a binding partner polypeptide. Binding of the labeled binding partner polypeptide to the immobilized SOCS-8 is measured by determining the extent to which the labeled binding partner polypeptide is immobilized as a result of a specific binding interaction. Such specific binding may be reversible, or may be optionally irreversible if a cross-linking agent is added in appropriate experimental conditions. Agents that inhibit or augment the formation of bound complexes as compared to a control binding reaction lacking agent are thereby identified as SOCS-8-modulating agents and are candidate therapeutic agents.

In one variation, the binding assay is performed in vivo in a cell, such as a yeast cell (e.g., Saccharomyces), and agents which inhibit intermolecular binding between a SOCS-8 protein and a binding partner polypeptide are identified as SOCS-8-modulating agents. For example, the in vivo screening assay is optionally a yeast two-hybrid system wherein the yeast cells express: (1) a first fusion protein comprising SOCS-8 and a first transcriptional regulatory protein sequence (e.g., GAL4 activation domain), (2) a second fusion protein comprising a binding partner polypeptide and a second transcriptional regulatory protein sequence (e.g., GAL4 DNA-binding domain), and (3) a reporter gene (e.g., beta-galactosidase, an auxotroph complementing gene) which is transcribed when an intermolecular complex comprising the first fusion protein and the second fusion protein is formed. If a functional SOCS-8: binding partner polypeptide complex forms, such as in a control assay lacking agent, the cell expresses the reporter gene which can be detected. Agents which inhibit or augment formation of functional SOCS-8: binding partner polypeptide complexes (and thus reporter gene expression) are thereby identified as SOCS-8-modulating agents and candidate drugs and commercial cell culture reagents and cell preservatives and the like.

In another embodiment, the physical interaction of the bound labeled complex with the surface is reported, such as where the surface is a fluor or scintillant and the label in the bound labeled complex emits radiation suitable for activating the fluor or scintillant of the surface; light emitted from the surface reports the relative amount of bound labeled complex. A suitable system is the scintillation proximity assay (Amersham), wherein the unlabeled component is bound to a fluor-containing bead. Alternative systems include the “Flash Plate” system (LKB).

In another variation, immobilization is not required; the SOCS-8 polypeptide is labeled with a first fluor which absorbs radiation (particle or wave) and emits phosphorescent or fluorescent light at a first wavelength, the binding partner polypeptide is labeled with a second fluor which absorbs radiation at said first wavelength and thereby emits fluorescent or phosphorescent radiation at a second wavelength. The labeled SOCS-8 polypeptide and the binding partner are incubated under suitable binding conditions, and at suitable reactant concentrations whereby the amount of radiation of the second wavelength is approximately proportional to the amount of bound SOCS-8/binding partner complexes, and excited with radiation of the firstwavelength (or particle type) and the amount of emitted radiation of the second wavelength is detected. The relative amount of radiation of the second wavelength reports the relative amount of bound DNA SOCS-8/binding partner complexes. An example of suitable system is a dye-dye transfer system (Packard)

It should be recognized, of course, that these assays are mentioned by way of example only and that these methods might be modified or that other methods of assaying SOCS activity would be apparent to one skilled in the art.

The invention also comprehends high throughput screening (HTS) assays to identify compounds that interact with or inhibit or enhance the biological activity (i.e., inhibit activity, binding activity, etc.) of a SOCS-8 polypeptide. HTS assays permit screening of large numbers of compounds in an efficient manner. Cell-based HTS systems are contemplated to investigate SOCS-8: binding partner interactions. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and the SOCS-8 polypeptide.

Typical examples of therapeutic agents based on the above presently described molecules include, but are not limited to, defective (either engineered or naturally occurring) forms of the proteins that associate with the protein complexes, inhibitory fragments of the proteins, wild type and altered genes that code for proteins that disrupt mammalian SOCS-8, small organic molecules, antisense nucleic acid sequences, oligonucleotides that inhibit expression or activity via a triplex mechanism, peptides, aptameric oligonucleotides, and the like. More particularly, examples of engineered proteins may include, but are not limited to, proteins that comprise inactivating mutations in conserved active sites (e.g., ATP binding motifs, DNA or protein binding domains, catalytic sites, etc.), fusion proteins that comprise at least one inhibitory domain, and the like.

The above agents may be obtained from a wide variety of sources. For example, standard methods of organic synthesis may be used to generate small organic molecules that specifically disrupt the relevant protein protein interactions. In addition, combinatorial libraries comprising a vast number of compounds (organic, peptide, or nucleic acid, reviewed in Gallop et al. 1994, J. Med. Chem. 37(9):1233-¹²⁵I; Gordon et al., 1994, J. Med. Chem. 37(10):1385-1401; and U.S. Pat. No. 5,424,186 all of which are herein incorporated by reference) may be screened for the ability to bind and inhibit protein protein interactions involved in SOCS-8 function.

Mutations in the SOCS-8 gene that result in loss of normal function of the SOCS-8 gene product underlie SOCS-8-related human disease states. The invention comprehends gene therapy to restore SOCS-8 activity to treat those disease states. Delivery of a functional SOCS-8 gene to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp.25-20 (1998). For additional reviews of gene therapy technology see Friedmann, Science, 244: 1275-1281 (1989); Verma, Scientific American: 68-84 (1990); and Miller, Nature, 357: 455-460 (1992). Alternatively, it is contemplated that in other human disease states, preventing the expression of or inhibiting the activity of SOCS-8 will be useful in treating the disease states. It is contemplated that antisense therapy or gene therapy could be applied to negatively regulate the expression of SOCS-8.

Additional features and variations of the invention will be apparent to those skilled in the art from the entirety of this application, including the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

It will be clear that the invention may be practiced otherwise than as particularly described in the foregoing description and examples.

Numerous modifications and variations of the present invention are possible in light of the above teachings and, therefore, are within the scope of the invention.

The entire disclosure of all publications cited herein are hereby incorporated by reference.

What is claimed is:

1. An isolated nucleic acid molecule comprising a polynucleotide having a nucleotide sequence encoding a SOCS-8 polypeptide having the amino acid sequence of SEQ ID NO:2

2. The isolated nucleic acid molecule of claim 1, wherein said polynucleotide comprises the nucleotide sequence of SEQ ID NO: 1

3. An isolated nucleic acid molecule encoding a SOCS-8 polypeptide comprising a polynucleotide that hybridizes under stringent conditions to a polynucleotide having the nucleotide sequence set forth in SEQ ID NO: 1

4. An isolated nucleic acid molecule encoding a SOCS-8 polypeptide comprising a polynucleotide having a sequence at least 95% identical to a polynucleotide having the nucleotide sequence set forth in SEQ ID NO: 1

5. A vector comprising a nucleic acid molecule of claim 1.

6. The vector of claim 5, wherein said nucleic acid molecule of claim 1 is operably linked to a promoter for the expression of a human SOCS-8 polypeptide.

7. A host cell comprising the vector of claim 5.

8. The host cell of claim 7, wherein said host is a eukaryotic host.

10. The host cell of claim 7, wherein said host cell is a baculovirus cell.

11. The host cell of claim 7 wherein said host cell is a yeast cell.

12. The host cell of claim 11 wherein said host cell is a Saccharomyces cerevisea

13. A method of obtaining a SOCS-8 polypeptide comprising culturing the host cell of claim 7 and isolating said SOCS-8 polypeptide.

14. A SOCS-8 polypeptide produced by the method of claim 13.

15. An isolated SOCS-8 polypeptide comprising an amino acid sequence of a SOCS-8 polypeptide having the amino acid sequence of SEQ ID NO:2 and conservative substitutions therein;

16. The SOCS-8 polypeptide of claim 15, wherein the isolated SOCS-8 polypeptide of 15 is expressed from an isolated nucleic acid molecule comprising a polynucleotide having the nucleotide sequence SEQ ID NO: 1

16. An isolated SOCS-8 polypeptide expressed from an isolated nucleic acid molecule comprising a polynucleotide having a sequence at least 95% identical to a polynucleotide having the nucleotide sequence of SEQ ID NO: 1;

17. A isolated SOCS-8 polypeptide comprising the amino acid sequence set forth in SEQ ID NO: 2, or a fragment thereof comprising an epitope specific to said polypeptide.

18. An antibody specific for the SOCS-8 polypeptides of claims 14-17.

19. The antibody of claim 18 which is a monoclonal antibody.

20. A hybridoma that produces an antibody according to claim 19.

22. An antibody according to claim 19 that is a humanized antibody.

23. A cell-free composition comprising polyclonal antibodies, wherein at least one of said antibodies is an antibody according to claim 18.

24. A method for identifying agents that modulate the propensity of a SOCS-8 polypeptide to associate with a binding partner polypeptide comprising
(a) contacting said SOCS-8 polypeptide and said binding partner polypeptide in the presence and absence of a test agent;
(b) determining the propensity of said SOCS-8 polypeptide to associate with a said binding partner polypeptide in the presence and absence of the test agent; and
(c) comparing the propensity of said SOCS-8 polypeptide to associate with said binding partner polypeptide in the presence of the test agent with the propensity of said SOCS-8 polypeptide to associate with a said binding partner polypeptide in the absence of the test agent to identify an agent that modulates the propensity of said SOCS-8 polypeptide to associate with a said binding partner polypeptide, wherein an agent which alters the propensity of said SOCS-8 polypeptide to associate with a said binding partner polypeptide is a modulator

25. A method according to claim 24, wherein the SOCS-8 polypeptide is a recombinant polypeptide purified and isolated from a cell transformed or transfected with a polynucleotide comprising a nucleotide sequence that encodes the polypeptide.

26. A method according to claim 24,
wherein the SOCS8 polypeptide is expressed in a cell transformed or transfected with a polynucleotide comprising a nucleotide sequence that encodes the polypeptide, wherein the contacting comprises growing the cell in the presence and absence of the test agent.

27 The method of claim 24, wherein said agent increases activity.

28. The method of claim 24, wherein said agent decreases activity.

29. A method for detecting the presence of a SOCS-8 polypeptide comprising the step of contacting said polypeptide with an antibody specific for said polypeptide, under conditions wherein the antibody binds the enzyme.
